(19) United States
(12) Patent Application Publication
(10) Pub. No.: US 2008/0214537 A1
(43) Pub. Date: Sep. 4, 2008

(54) BRIDGED PHENANTHRIDINES

(75) Inventors: Janet L. Ralbovsky, Shelton, CT (US); R. Paul Beckett, Yorktown Heights, NY (US)

Correspondence Address: CARA THERAPEUTICS, INC.;Intellectual Property Department, One Parrott Drive, Shelton, CT, 06484-4733, US

(73) Assignee: CARA THERAPEUTICS, INC., SHELTON, CT (US)

(21) Appl. No.: 12/040,565

(22) Filed: Feb. 29, 2008

Related U.S. Application Data
(60) Provisional application No. 60/904,644, filed on Mar. 2, 2007.

(51) Int. Cl. A61K 31/5377; C07D 221/22; A61K 31/439; C07D 413/12

(52) U.S. Cl. 514/232.8; 546/74; 514/289; 544/125

(57) ABSTRACT

Bridged phenanthridine compounds having the structure of formula I or of formula II are provided:

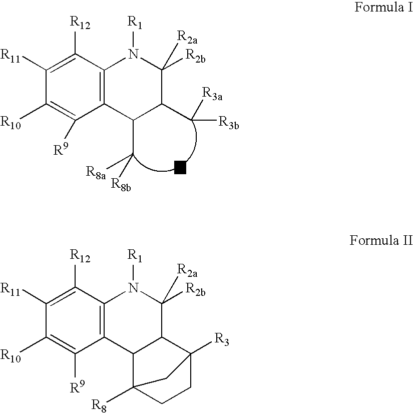

These compounds are useful as cannabinoid receptor ligands and can be prepared as pharmaceutical compositions for the prophylaxis or treatment of a variety of diseases, disorders and conditions including inflammatory pain, visceral pain, postoperative pain, cancer pain, neuropathic pain, musculoskeletal pain, dysmenorrhea, menstrual pain, migraine, headache as well as inflammatory diseases such as rheumatoid arthritis, systemic lupus erythematosus, Crohn's disease, psoriasis, eczema, multiple sclerosis, diabetes and thyroiditis. The compounds can also be used in the treatment of skin disorders, lung disorders, ophthalmic disorders, gastrointestinal disorders, cardiovascular disorders, as well as neurodegenerative, neuroinflammatory and certain psychiatric disorders.

This application claims the benefit of U.S. provisional patent application Ser. No. 60/904,644 filed Mar. 2nd, 2007 the specification of which is hereby incorporated by reference in its entirety.

The present invention provides in certain embodiments compounds having the general structure of formula I as shown below:

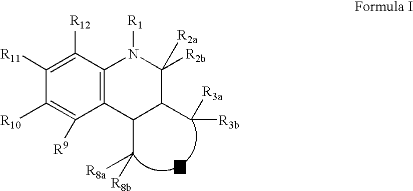

The invention additionally provides pharmaceutically acceptable salts, esters, solvates, stereoisomers, or racemates of compounds of Formula I.

In the compounds of Formula I, the moiety B is a bond or a 1, 2, 3, or 4-carbon atom chain which completes a four to eight-membered saturated, singly unsaturated, or doubly unsaturated carbocyclic ring, such as, for instance and without limitation, cyclobutyl, cyclopentyl, cyclopentenyl, cyclohexyl, cycloheptyl, and cyclooctyl moieties which are fused with the piperidinyl ring of Formula I. Singly unsaturated carbocyclic rings refer to rings having a single double bond and doubly unsaturated carbocyclic rings refer to rings having two non-adjacent double bonds. Each of the ring atoms in B may be optionally substituted with one or more substituents independently selected from H, C₁-C₄ alkyl, C₁-C₄ alkoxy, C₁-C₄ haloalkyl, C₁-C₄ haloalkoxy, fluoro, chloro, cyano, hydroxyl, C₁-C₄ hydroxy alkyl. Additionally, in those embodiments where B completes a seven or eight-membered ring, two nonadjacent carbon atoms in the chain B can be bridged by a methylene or an ethylene moiety.

The R₁ group is H, C₁-C₅ straight, branched or cyclic alkyl, —COR₁₃, —SO₂R₁₃, benzyl, or alkyl or halo substituted benzyl and R₁₃ is —OH, C₁-C₄ alkyl, which can be a straight chain or branched.

R₂ₐ is -L-R₁₄ such that the optional linker L is —Xₜ(CO)ₘXₚ(CH₂)ₙY_{q}— and X is O or NH, Y is O or S, m is 0or 1, n is 0, 1, or 2, p is 0 or 1, q is 0 or 1, and t is 0 or 1; wherein, if p and t are both 1, then m is 1, q is zero and X is NH. R₁₄ is optionally substituted aryl, optionally substituted heteroaryl, optionally substituted cycloalkyl, optionally substituted saturated heterocyclyl, optionally substituted straight or branched C₁-C₈ alkyl, or optionally substituted straight or branched C₁-C₈ heteroalkyl. In certain embodiments, when R₁₄ comprises aryl or heteroaryl, then the aryl or heteroaryl moiety is either a single 5 or 6-membered ring or a fused bicyclic moiety. R_{2b} is H or methyl.

In certain embodiments R₃ₐ and R₈ₐ complete a bridge across the ring completed by B in Formula I, such that either R₃ₐ and R₈ₐ taken together form a methylene or ethylene bridge or one of R₃ₐ and R₈ₐ is H and the other forms a methylene or ethylene bridge to a carbon atom in the chain B. The bridge carbons of such a bridge may be unsubstituted or substituted with methyl, chloro, or fluoro; in certain embodiments a bridge carbon may be geminal dimethyl substituted.

In other embodiments R₃ₐ and R₈ₐ do not complete a bridge and instead are each selected independently from the group consisting of H, C₁-C₄ alkyl, C₁-C₄ alkoxy, C₁-C₄ haloalkyl, C₁-C₄ haloalkoxy, fluoro, chloro, cyano hydroxyl, C₁-C₄ hydroxy alkyl. In other embodiments one or both of R₃ₐ and R₈ₐ is a bond forming a second ring bond with an adjacent ring carbon atom thereby completing a double bond in the ring, such as, for instance, the double bond in a cyclopentenyl moiety.

Rhd 3b and R_{8b} are each independently selected from the group consisting of H and methyl.

R₉, R₁₁, and R₁₂ are each independently selected from the group consisting of H, halo, nitro, cyano, hydroxyl, amino, C₁₋₄ alkyl, C₁₋₄ alkoxy, C₁₋₄ haloalkoxy, C₁₋₄ haloalkyl, trihalomethoxy, trihalomethyl, and cyclopropyl.

R₁₀ is a group having an optional methylene, ethylene or propylene linker and a carbonyl, ester, thionyl, amine, amide, reverse amide, thioamide, reverse thioamide, sulfonyl, sulfoxyl, thioether, sulfonamide, reverse sulfonamide, urea, or a thiourea moiety as a linker to R₁₅ or, alternatively, having a formula chosen from the following: —(CH₂)ᵣ—CO—(CH₂)ᵣR₁₅, —(CH₂)ᵣ—COO—(CH₂)ᵣR₁₅, —(CH₂)ᵣ—CS—(CH₂)ᵣR₁₅, —(CH₂)ᵣN(R₁₆)((CH₂)ᵣR₁₇), —(CH₂)ᵣ—CO—N(R₁₆)((CH₂)ᵣR₁₇), —(CH₂)ᵣNH—CO—(CH₂)ᵣR₁₅, —CH₂)ᵣ—CS—N(R₁₆)((CH₂)ᵣR₁₇), —(CH₂)ᵣNH—CS—(CH₂)ᵣR₁₅, —(CH₂)ᵣSO₂—(CH₂)ᵣR₁₅, —(CH₂)ᵣSO—(CH₂)ᵣR₁₅, —(CH₂)ᵣS—(CH₂)ᵣR₁₅, —(CH₂)ᵣSO₂—N(R₁₆)((CH₂)ᵣR₁₇), —(CH₂)ᵣNH—SO₂—(CH₂)ᵣR₁₅, —(CH₂)ᵣNH—CO—N(R₁₆)((CH₂)ᵣR₁₇), and —(CH₂)ᵣNH—CS—N(R₁₆)((CH₂)ᵣR₁₇). Additionally R₁₀ can be a group having an optional methylene, ethylene or propylene linker coupled to a 5-6 membered heteroaryl ring optionally substituted with R₁₅. In R₁₀ each r is independently selected from 0, 1, 2, and 3; R₁₅ is H or optionally substituted straight or branched C₁-C₈ alkyl, optionally substituted straight or branched C₁-C₈ heteroalkyl, optionally substituted C₃-C₈ cycloalkyl, optionally substituted 5-7 membered cycloheteroalkyl, optionally substituted aryl, or optionally substituted 5-6 membered or bicyclic heteroaryl.

R₁₆ and R₁₇ may be taken together to form an optionally substituted saturated heterocyclyl with the N to which they are bonded in which case r is zero, such that the optional alkylene linker between such N atom and R₁₇ is null. Alternatively, R₁₆ and R₁₇ can be each independently selected from the group consisting of substituted straight chain or branched C₁-C₈ alkyl, optionally substituted straight or branched C₁-C₈ heteroalkyl, optionally substituted C₃-C₈ cycloalkyl, optionally substituted 5-7 membered cycloheteroalkyl, optionally substituted aryl, optionally substituted 5-6 membered and bicyclic heteroaryl.

In certain embodiments of Formula I, when R₂ₐ is unsubstituted phenyl, when R₁, R_{2b}, R_{3b}, R_{8b}, R₉, R₁₁, R₁₂ are all H, when R₂ₐ, R₈ₐ and B taken together complete an unsubstituted norbornanyl moiety, and when R₁₀ is —CO—N R₁₆R₁₇, then R₁₆ is —(CH₂)ₛR₁₈ wherein R₁₈ is optionally substituted aryl or cyclopropyl, and s is 1, 2, or 3 and R₁₇ is H.

In certain embodiments of Formula I when R₁₀ is —COOH, and R₁, R_{2b}, R_{3b}, R_{8b}, R₉, R₁₁, R₁₂ are all H, wherein R₂ₐ, R₈ₐ and B taken together complete an unsubstituted norbornanyl moiety, and wherein m, n, p, q, and t are all zero, then R₁₄ is not phenyl.

In certain embodiments of Formula I, R₃ₐ, R₈ₐ and B are taken together to complete an optionally substituted, bridged 6, 7 or 8-membered ring. In certain of such embodiments the bridge is a methylene moiety; in other such embodiments the bridge is an ethylene moiety. In certain embodiments of Formula I, R₃ₐ, R₈ₐ and B taken together complete an optionally substituted norbornanyl moiety.

In certain other embodiments of Formula I, R₃ₐ and R₈ₐ are H and do not form a bridge and B completes a cyclopentenyl moiety.

In certain embodiments, the ring nitrogen of Formula I is unsubstituted, i.e., R₁ is H. In certain embodiments, the ring nitrogen of Formula I is substituted with C₁-C₅ alkyl. In certain embodiments, the ring nitrogen of Formula I is substituted with a carbonyl moiety —COR₁₃. In certain other embodiments, the ring nitrogen of Formula I is substituted with a sulfonyl moiety —SO₂R₁₃.

In certain embodiments of Formula I, the phenyl ring substituents R₉, R₁₁, and R₁₂ are all H. In other embodiments of Formula I, the linker L in R₂ₐ is null or alkylene such that m, p, q, and t are zero. In particular embodiments, n is an integer. In other particular embodiments n is zero.

In certain other embodiments of Formula I, R₁₄ is optionally substituted aryl or optionally substituted heteroaryl. In still other embodiments of Formula I, R₁₄ is optionally substituted phenyl. In alternative embodiments of Formula I, R₁₄ is optionally substituted heteroaryl. In other alternative embodiments of Formula I, R₁₄ is optionally substituted 5 or 6-membered heteroaryl, which in certain particular embodiments is optionally substituted pyrimidinyl, pyridinyl, pyrazinyl, thiazolyl, furanyl, thiophenyl, oxazolyl, or imidazolyl. In certain other embodiments of Formula I, R₁₄ is optionally substituted straight or branched C₁-C₆ alkyl.

In particular embodiments of Formula I all integers designated as “r”0 in R₁₀ are zero such that R₁₀ does not contain fully saturated alkylene linkers. In other embodiments of Formula I, at least one r is not zero; for example when r is 1 or 2 so as to provide a methylene or an ethylene linker, respectively, in R₁₀ linking to R₁₅ or R₁₆.

In certain embodiments of Formula I, R₁₀ is a sulfonyl-linked R₁₅, of the formula —SO₂R₁₅ or a “reverse” amide of the formula —NH—CO—R₁₅.

In certain other embodiments of Formula I, R₁₀ is a sulfonamide of the formula —SO₂—NR₁₆R₁₇.

In yet other embodiments of Formula I, R₁₀ is a reverse sulfonamide of the formula —(CH₂)ᵣNH—SO₂R₁₅. In still other certain embodiments of Formula I, R₁₀ is a urea of the formula —(CH₂)ᵣNH—CO—NR₁₆R₁₇.

In particular embodiments of Formula I, R₁₀ is an optional C₁-C₃ alkylene linker coupled to a 5-6 membered heteroaryl ring which can be substituted with R₁₅.

Also provided in the present invention are compounds having the structure of formula II:

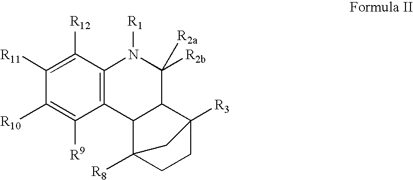

and pharmaceutically acceptable salts, esters, solvates, stereoisomers and racemates thereof, wherein R₁ is chosen from H, C₁-C₅ straight, branched or cyclic alkyl, —COR₁₃, or —SO₂R₁₃, wherein R₁₃ is C₁-C₃ straight or branched alkyl; R₂ₐ is -L-R₁₄ where L is —(NH)ₚ(CH₂)ᵣY_{q}— and Y is carbonyl or thionyl, p and q are each independently 0 or 1; wherein R₁₄ is chosen from optionally substituted aryl, optionally substituted cycloalkyl, optionally substituted heterocyclyl, optionally substituted straight or branched C₁-C₄ alkyl, or optionally substituted straight or branched C₁-C₄ heteroalkyl; and when R₁₄ includes aryl or heterocyclyl moiety, such aryl or heterocyclyl moiety is either a single 5- or 6-membered ring or a fused bicyclic moiety.

In Formula II, R_{2b} is H or methyl; R₃ and R₈ are each independently chosen from hydrogen and C₁-C₃ alkyl; R₉, R₁₁ and R₁₂ are each independently chosen from hydrogen, halo, cyano, hydroxyl, amino, and C₁₋₄ alkyl; R₁₀ is chosen from —(CH₂)ᵣ—COO—(CH₂)ᵣ—R₁₅, —(CH₂)ᵣN(R₁₆)((CH₂)ᵣ—R₁₇), —(CH₂)ᵣR₁₆—(CH₂)ᵣ—R₁₇, —(CH₂)ᵣ—CO—N(R₁₆)((CH₂)ᵣ—R₁₇), —(CH₂)ᵣNH—CO—(CH₂)ᵣ—R₁₅, —(CH₂)ᵣSO₂—(CH₂)ᵣ—R₁₅, —(CH₂)ᵣSO—(CH₂)ᵣ—R₁₅, —(CH₂)ᵣSO₂—N(R₁₆)((CH₂)ᵣR₁₇), —(CH₂)ᵣSO₂—NR₁₆—CO—((CH₂)ᵣ—R₁₇), —(CH₂)ᵣNH—SO₂—(CH₂)ᵣ—R₁₅, —(CH₂)ᵣNH—CO—N(R₁₆)((CH₂)ᵣ—R₁₇), —(CH₂)ᵣNH—CS—N(R₁₆)((CH₂)ᵣ—R₁₇), (CH₂)ᵣN—(SO₂R₁₆)(SO₂R₁₇), —CO—NH—(CH₂)ᵣ—(C₃-C₆ cycloalkyl) and —CO—NH—(CH₂)ᵣ-(aryl); wherein each r is independently chosen from 0, 1, 2, and 3; and wherein R₁₅ is chosen from hydrogen, optionally substituted straight or branched C₁-C₆ alkyl, optionally substituted straight or branched C₁-C₆ heteroalkyl, optionally substituted C₃-C₈ cycloalkyl, optionally substituted 5-7 membered heterocyclyl, optionally substituted aryl and optionally substituted 5-6 membered or bicyclic heteroaryl. The moieties R₁₆ and R₁₇ are either taken together to form an optionally substituted heterocyclyl or are each independently chosen from hydrogen, substituted straight or branched C₁-C₆ alkyl, optionally substituted straight or branched C₁-C₆ heteroalkyl, optionally substituted C₃-C₈ cycloalkyl, optionally substituted 5, 6 or 7-membered heterocyclyl, optionally substituted aryl and optionally substituted 5 or 6-membered heteroaryl.

In Formula II Rio cannot be a carboxylate moiety. Further, when R₂ₐ is unsubstituted phenyl, and R₁, R_{2b}, R_{3b}, R_{8b}, R₉, R₁₁ and R₁₂ are all hydrogen, and R₁₀ is —CO—NHR₁₆, then the moiety R₁₆ is —(CH₂)ₛR₁₈, wherein R₁₈ is optionally substituted aryl or cyclopropyl and s is 1, 2, or 3.

In certain embodiments of Formula II when, R₁ is hydrogen or —SO₂R₁₃: then R_{2b}, R₃, R₈, R₉, R₁₁ and R₁₂ are each H; and R₁₀ is chosen from —(CH₂)ᵣ—COO—(CH₂)ᵣ—R₁₅, —(CH₂)ᵣN(R₁₆)((CH₂)ᵣ—R₁₇), —(CH₂)ᵣR₁₆—(CH₂)ᵣ—R₁₇, (CH₂)ᵣ—CO—N(R₁₆)((CH₂)ᵣ—R₁₇), —(CH₂)ᵣNH—CO—(CH₂)ᵣ—R₁₅, —(CH₂)ᵣSO₂—(CH₂)ᵣ—R₁₅, —(CH₂)ᵣSO₂—N(R₁₆)((CH₂)ᵣ—R₁₇), —(CH₂)ᵣSO₂—N(R₁₆)—CO—((CH₂)ᵣ—R₁₇), —(CH₂)ᵣNH—SO₂—(CH₂)ᵣ—R₁₅, —(CH₂)ᵣNH—CO—N(R₁₆)((CH₂)ᵣ—R₁₇), —(CH₂)ᵣNH—CS—N(R₁₆)((CH₂)ᵣ—R₁₇), —CO—NH—(CH₂)ᵣ—C₃-C₆ cycloalkyl and —CO—NH—(CH₂)ᵣ-aryl; and R₁₄ is chosen from optionally substituted straight or branched C₁-C₃ alkyl, optionally substituted aryl, optionally substituted heteroaryl, and optionally substituted heterocyclyl.

In certain other embodiments of Formula II the operators in, p, q and t are each 0. In certain other particular embodiments of Formula II the operator n is 1 or 2; in certain other particular embodiments n is 0. In other particular embodiments, each instance of the operator, r is zero.

In particular embodiments of Formula II, R₁₄ is optionally substituted aryl. In other particular embodiments R₁₄ is optionally substituted 5 or 6-membered heteroaryl. In still other particular embodiments R₁₀ is the moiety —(CH₂)ᵣSO₂—N(R₁₆)((CH₂)ᵣ—R₁₇) or the moiety —(CH₂)ᵣNH—CO—(CH₂)ᵣ—R₁₅. In alternative embodiments, R₁₀ is chosen from —(CH₂)—COO—(CH₂)ᵣ—R₁₅, —(CH₂)ᵣN(R₁₆)((CH₂)ᵣ—R₁₇), —(C₂)ᵣR₁₆—(CH₂)ᵣ—R₁₇, —(CH₂)ᵣ—CO—N(R₁₆)((CH₂)ᵣ—R₁₇), —(CH₂)ᵣSO₂—(CH₂)ᵣ—R₁₅, and —(CH₂)ᵣSO₂—N(R₁₆)—CO—((CH₂)ᵣ—R₁₇). In alternative embodiments, R₁₀ is chosen from —(CH₂)ᵣNH—SO₂—(CH₂)ᵣ—R₁₅, —(CH₂)ᵣNH—CO—N(R₁₆)((CH₂)ᵣ—R₁₇), —(CH₂)ᵣNH—CS—N(R₁₆)((CH₂)ᵣ—R₁₇), (CH₂)ᵣN—(SO₂R₁₆(SO₂R₁₇), —CO—NH—(CH₂)ᵣ—(C₃-C₆ cycloalkyl) and —CO—NH—(CH₂)ᵣ-(aryl).

The present invention also provides compounds having the structure of Formula II, wherein in certain embodiments the compounds have an EC₅₀ of less than about 1 μM for a mammalian CB2 receptor. In other embodiments, the compounds have an EC₅₀ of less than about 500 μM for a mammalian CB2 receptor. In still other embodiments, the compounds have an EC₅₀ of less than about 100 nM for a mammalian CB2 receptor. In other embodiments, the compounds have an EC₅₀ of less than about 75 nM for a mammalian CB2 receptor. In yet other embodiments, the compounds have an EC₅₀ of less than about 20 nM for a mammalian CB2 receptor. In certain other embodiments, the compounds have an EC₅₀ of less than about 10 nM for a mammalian CB2 receptor. In certain embodiments, the compounds have an EC₅₀ of less than about 1.0 nM for a mammalian CB2 receptor. The mammalian CB2 receptor can be any mammalian CB2, such as for instance and without limitation, a mouse CB2 receptor, a rat CB2 receptor or in a particular aspect, the mammalian CB2 receptor can be a CB2 receptor of a primate, such as a human.

The present invention also provides compounds of the structure of Formula II, which in certain embodiments have an EC₅₀ of greater than about 100 nM for a mammalian CB1 receptor. In other embodiments, the compounds have an EC₅₀ of greater than about 1.0 μM for a mammalian CB1 receptor. In still other embodiments, the compounds have an EC₅₀ of greater than about 10 μM for a mammalian CB1 receptor. In still other embodiments, the compounds have an EC₅₀ of greater than about 100 μM for a mammalian CB1 receptor. The mammalian CB1 receptor can be any mammalian CB1, such as for instance and without limitation, a mouse CB1 receptor, a rat CB1 receptor or in a particular aspect, the mammalian CB1 receptor can be a CB1 receptor of a primate, such as a human.

DEFINITIONS: As used in this specification the following terms have the definitions listed below:

“Halo” means fluoro, chloro, bromo or iodo. Fluoro and chloro groups are preferred halo substituents. Halo alkyl and halo alkoxy means an alkyl or alkoxy group having one or more H atoms replaced with a halo group and includes perhalo substituted groups.

“Alkyl” means a linear or branched carbon chain, such as, but without limitation, methyl, ethyl, propyl, isopropyl, n-butyl, sec-butyl, iso-butyl, tert-butyl, pentyl, hexyl, heptyl, octyl, and their branched chain isomers. The term “alkenyl” means a linear or branched carbon chain with one or more double bonds, such as, but without limitation, vinyl, allyl, isopropenyl, pentenyl, hexenyl, heptenyl, 1-propenyl, 2-butenyl, 2-methyl-2-butenyl. “Alkynyl” means a linear or branched carbon chain with one or more triple bonds, such as for instance, ethynyl, propynyl, 3-methyl-1-pentynyl and 2-heptynyl. As used herein “cycloalkyl” means a monocyclic, bicyclic or bridged saturated carbocyclic ring, each ring having from 3-8 carbon atoms. Norbornane and adamantane are examples of such cycloalkyl moieties that are bicyclic or bridged saturated carbocyclic rings. The term “cycloalkyl” also includes a monocyclic ring fused to an aryl or heteroalkyl substituent. Cycloalkyl substituents include but are not limited to cyclopropyl, cyclobutyl, cyclopentyl, cyclohexyl, cycloheptyl and tetrahydronaphthyl.

“Heteroalkyl” means a linear or branched chain comprising carbon atoms and at least one heteroatom (N, S, or O) such as ethers, thioethers and amines. Cycloheteroalkyl means a monocyclic or bicyclic ring having at least one ring heteroatom.

“Alkylene” means an alkyl diradical bonded to other moieties in two locations such as methylene (—CH₂—), ethylene (—CH₂CH₂—), etc. Alkylene moieties can form linkers interposed between two moieties attached at any two carbon atoms of the alkylene moiety.

“Alkoxy” means an alkyl moiety having an ether linkage such as methoxy, ethoxy, etc. Hydroxy alkyl means an alkyl moiety substituted with one or more hydroxyl groups, e.g., 2-hydroxy ethyl.

Where a term such as “alkyl”, “alkoxy”, or “cycloalkyl”, etc. is modified with a specified number of carbon atoms or a range of number of carbon atoms, e.g., C₁-C₄ alkyl, such moiety has a number of carbon atoms within the specified range, i.e., in this example 1-4, corresponding to methyl, ethyl, propyl, isopropyl, n-butyl, sec-butyl, or t-butyl.

“Aryl” includes a monocyclic or bicyclic aromatic ring including only carbon atoms in the ring. “Aryl” also includes an aryl group fused to a monocyclic cycloalkyl or monocyclic cycloheteroalkyl group. Aryl groups include phenyl, naphthyl, quinolinyl, tetrahydroquinolinyl, benzofuranyl and dihydrobenzopyranyl.

“Heteroaryl” means a monocyclic, bicyclic or tricyclic aromatic ring containing at least one heteroatom with each ring containing 5 or 6 atoms. The heteroatom(s) can be independently selected from N, O or S. Examples of monocyclic heteroaryl groups include thiophenyl, pyrolyl, pyrolinyl, furanyl, axazolyl, thiazolyl, imidazolyl, imidazolinyl, imidazolidinyl, pyrazolyl, triazolyl, thiadiazolyl, pyranyl, pyridyl, pyridazinyl, pyrimidinyl, prazinyl and triazinyl. Examples of bicyclic heteroaryl groups include indolyl, benzofuranyl, benzothiophenyl, benzthiazolyl, purinyl, quinolinyl, isoquinolinyl, quinazolinyl and pteridinyl. Examples of tricyclic heteroaryl groups include carbazolyl, phenothiazinyl and phenoxazinyl.

“Heterocyclyl” means a group of one or more fused rings, wherein at least one ring includes at least one heteroatom. The heterocyclyl group can, but need not include one or more unsaturated bonds, and can in certain embodiments include one or more aromatic rings. Thus, as used in this specification, the term “heterocyclyl” includes “heteroaryl” groups as well as unsaturated non-aromatic ring-containing groups and fully saturated cyclic groups having at least one heteroatom.

Where a moiety is herein described as optionally substituted, such moiety may be unsubstituted, singly substituted or multiply substituted with the same or different substituents. Optional substituents can be, for example, an alkyl, alkenyl, alkynyl, cycloalkyl, cycloalkenyl, aryl, alkaryl, aralkyl, heteroaryl, heteroaralkyl, cycloheteroalkyl, haloalkyl, perhaloalkyl, alkoxy, carboxy, amino, amide, sulfonyl, sulfoxyl, sulfonamidyl, hydroxyl, halo, nitro, cyano, thio, or alkylthio group.

The term “bridged” compound as used herein refers to compounds having an alkylene bridge across the carbocycle formed by B in formula I. Thus, the compound “methylene bridged N-(cyclopropylmethyl)-6-phenyl-5,6,6a,7,8,9,10,10a-octahydro phenanthridine-2-carboxamide” refers to the compound having a methylene bridge across the saturated carbocycle of the phenanthridine (See Example 2d).

Compounds of the invention can have one or more asymmetric centers called chiral centers. In certain embodiments, the carbon atoms at the 2, 3 and 4 positions of the nitrogen-containing ring of compounds of formula I or of formula II can be chiral centers. These compounds can occur as racemic mixtures, single enantiomers, diastereomeric mixtures and individual diastereomers with respect to all or any subset of chiral centers of such compounds. All sterioisomeric forms of the compounds of formula I or of formula II are contemplated within the scope of the present invention.

In certain embodiments the compounds of the invention are agonists for a mammalian G protein-coupled receptor (GPCR), particularly a human GPCR. In particular embodiments, the compounds of the invention are agonists for a mammalian cannabinoid CB2 receptor, such as for instance, the human CB2 receptor. In certain embodiments, the compounds of the invention are full agonists for the human CB2 receptor. That is, when contacted with the human CB2 receptor at sufficiently high concentrations above the EC₅₀, these compounds are capable of fully inducing the activity of the receptor.

In other particular embodiments, the compounds of the invention are partial agonists for the human CB2 receptor. When contacted with the human CB2 receptor at concentrations above the EC₅₀, the maximum level of activity induced by these compounds is significantly below the maximal activity of the receptor induced by the full agonists. As used herein, compounds that showed induction of less than 80% of the maximal range of activity induced by CP55940 are designated as partial agonists.

In some embodiments, the compounds of the invention are selective agonists for a mammalian CB2 receptor and do not function as agonists for the CB1 receptor of that mammal. In particular embodiments, the compounds are selective agonists for the human CB2 receptor while having little or no agonist activity for the human CB1 receptor. In a particular embodiment, the compound of the invention is a salt, solvate, ester, stereoisomer, mixture of one or more stereoisomers, or a racemate of a compound having the structure of formula I. In another particular embodiment, the invention provides a pharmaceutically acceptable salt, solvate, ester, stereoisomer, mixture of one or more stereoisomers, or racemate of a compound having the formula of formula II.

Without wishing to be bound by theory, the compounds of the present invention are believed to be ligands for a mammalian CB2 receptor. In certain embodiments, the compounds of the present invention have an EC₅₀ of less than about 1 μM for a mammalian CB2 receptor. In other embodiments the compounds have an EC₅₀ of less than about 500 μM, while in other embodiments the compounds have an EC₅₀ of less than about 100 μM for a mammalian CB2 receptor, particularly the human CB2 receptor. In other embodiments, the compounds have an EC₅₀ of less than about 75 nM for the human CB2 receptor. In still other embodiments, the compounds have an EC₅₀ of less than about 50 nM for the human CB2 receptor. In yet other embodiments, the compounds have an EC₅₀ of less than about 20 nM for the human CB2 receptor. In still other embodiments, the compounds have an EC₅₀ of less than about 10 nM for the human CB2 receptor. In other embodiments, the compounds have an EC₅₀ of less than about 5 nM for the human CB2 receptor. In other particular embodiments, the compounds have an EC₅₀ of less than about 1.0 nM for the human CB2 receptor.

In one embodiment, the invention provides a pharmaceutical composition that includes a pharmaceutically effective amount of a compound of the invention. In a particular embodiment, the invention provides a pharmaceutical composition of the invention is effective for treating a disease condition or state addressable by modulating the activity of a cannabinoid CB2 receptor in a human or an animal in need thereof. In one aspect the disease condition or state is addressable by increasing the activity of the CB2 receptor with an agonist. In another aspect the disease condition or state is addressable by reducing the activity of the CB2 receptor with an antagonist.

In view of their ability to modulate the activity of the CB2 receptor, the compounds of the present invention can be used in the treatment of conditions and diseases or disorders that include, but are not limited to, inflammatory diseases such as rheumatoid arthritis, systemic lupus erythematosus, Crohn's disease, psoriasis, eczema, multiple sclerosis, diabetes and thyroiditis.

Certain compounds of the invention can also be used in the treatment of disorders such as, but are not limited to, pain (e.g. inflammatory pain, visceral pain, postoperative pain, cancer pain, neuropathic pain, musculoskeletal pain, dysmenorrhea, menstrual pain, migraine and headache).

Certain compounds of the invention can also be used in the treatment of skin disorders (e.g. sunburn, dermatitis, pruritis); lung disorders (e.g. chronic obstructive pulmonary disease, cough, asthma, bronchitis); ophthalmic disorders (e.g. glaucoma, retinitis, reinopathies, uveitis, conjunctivitis); gastrointestinal disorders (e.g. ulcerative colitis, irritable bowel syndrome, coeliac disease, inflammatory bowel disease, gastroesophageal reflux disease, organ transplant, nausea, emesis); cardiovascular disorders (e.g. stroke, cardiac arrest, atherosclerosis, myocardial ischemia); neurodegenerative, neuroinflammatory or psychiatric disorders (e.g. senile dementia, Alzheimer's disease, vascular dementia, amyotrophic lateral sclerosis, neuroinflammation, tinnitus); bladder disorders (e.g. bladder hyper-reflexia, cystitis) and cancer, such as for instance, lymphoblastic leukemia and lymphoma, acute myelogenous leukemia, chronic lymphocytic leukemia, glioma, skin cancer, breast cancer, prostate cancer, liver cancer, kidney cancer, lung cancer, pancreatic cancer.

In addition, certain compounds of the invention can be used to modulate bone formation and/or resorption for treating conditions including, but not limited to, ankylosing spondylitis, gout, arthritis associated with gout, osteoarthritis and osteoporosis.

Certain compounds of the invention can also be used for the treatment of neuropathic pain including, but not limited to diabetic neuropathy, fibromyalgia, lower back pain, sciatica, pain from physical trauma, cancer, amputation, toxins or chronic inflammatory conditions.

Compounds of the invention and their pharmaceutically acceptable salts can be administered in a standard manner, for example orally, parenterally, sublingually, dermally, transdermally, rectally, or via inhalation, or by buccal, nasal, ocular or otic administration.

### EXAMPLES

All reactions involving moisture sensitive compounds were carried out under an anhydrous nitrogen or argon atmosphere. All reagents were purchased from commercial sources and used without further purification. Normal phase chromatography was done on an ISCO CombiFlash Companion and reverse phase chromatography was done on a Waters AutoPurification System with 3100 Mass Detector. Mass spectra (MS) were determined on the Waters SQ Detector/3100 Mass Detector using electrospray techniques. Unless otherwise noted, the materials used in the examples were obtained from readily available commercial sources or synthesized by standard methods known to those skilled in the art of organic synthesis. Nuclear magnetic resonance spectra were recorded using a Jeol ECX 400 MHz spectrometer.

### Example 1

General scheme for synthesis of bridged phenanthridines of the invention. Final compounds of the invention can be isolated by filtration. Alternatively, the compounds can be isolated by normal, or reverse phase chromatography.

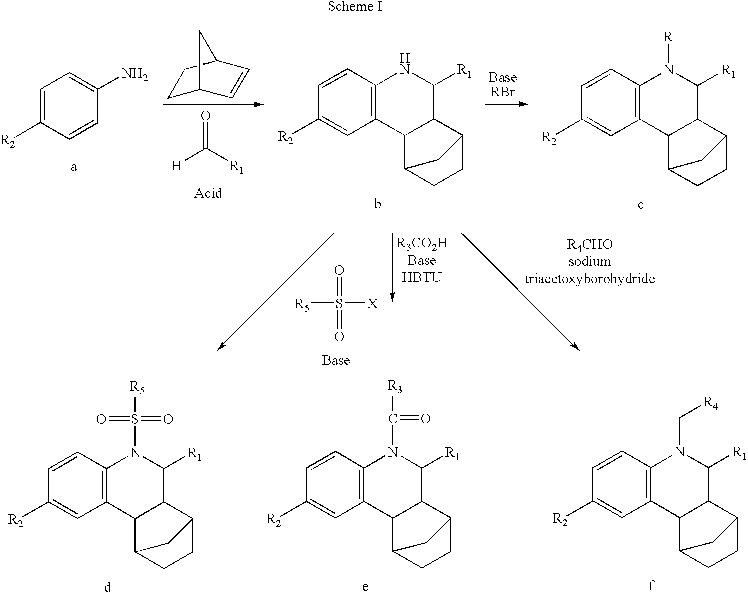

Certain groups of intermediates (1a and 1b) and groups of compounds of the present invention (1c, 1d, 1e and 1f) can be prepared according to the process outlined in the general synthetic schemes shown in Scheme I above.

### Example 2

### Synthesis of Compound (2d): Methylene bridged N-(cyclopropylmethyl)-6-phenyl-5,6,6a,7,8,9,10,10a-octahydrophenanthridine-2-carboxamide

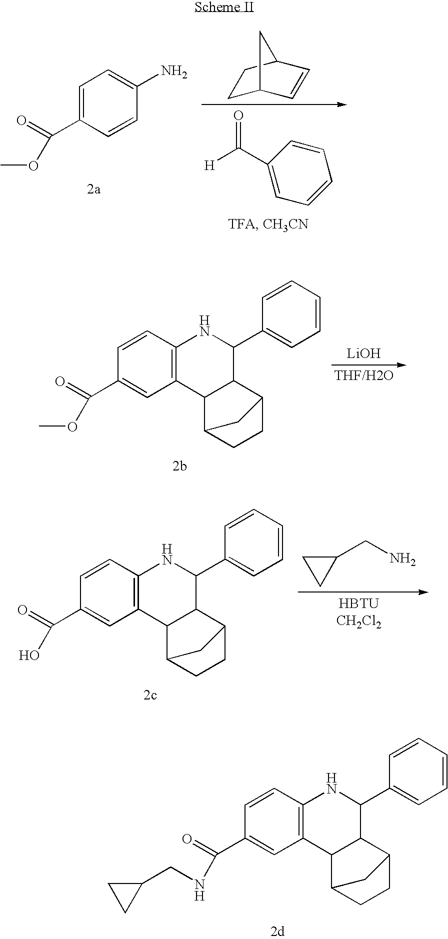

A. Synthesis of compound 2b: To commercially available 2a (0.30 g, 1.98 mmol) in acetonitrile (1 mL) was added benzaldehyde (0.23 ml, 2.34 mmol), norbornylene (0.40 g, 4.25 mmol) and trifluoroacetic acid (0.24 ml, 2.14 mmol). The reaction was stirred at room temperature for 16 hours. The desired product 2b precipitated out of solution and was collected via filtration to provide 0.40 g of a white solid.

B. Synthesis of compound 2c: To 2b (0.52 g, 1.56 mmol) in 3 ml THF (tetrahydrofuran) and 1 ml water was added lithium hydroxide (0.37 g, 15.6 mmol). The reaction stirred at room temperature for five days. Reaction was not complete so one equivalent of potassium hydroxide was added and the reaction was heated to 60° C. for 16 hours. Ethyl acetate was added and the organic layer was washed with water. The organic layer was concentrated to give the desired acid 2c which was used without further purification.

C. Synthesis of compound 2d. To 2c (0.050 g, 0.15 mmol) in methylene chloride (1 ml) was added cyclopropylmethanamine (0.022 g, 0.31 mmol), and HBTU (2-(1H-benzotriazole-1-yl)-1,1,3,3-tetramethyluronium hexafluorophosphate: 0.037 g, 0.15 mmol). The reaction stirred at room temperature for 16 hours. The reaction mixture was diluted with water and extracted with methylene chloride. The organics were dried over sodium sulfate and concentrated. The crude mixture was purified using normal phase chromatography using a 10-60% ethyl acetate/hexanes gradient to provide 3.7 mgs of 2d as a white solid. Mass spectrometry (MS) of the molecule ion yielded the following: MS: m/z 373.2 (MH+).

### Example 3

### Synthesis of Compound 3b: Bridged 6-phenyl-2-(piperidin-1-ylsulfonyl)-5,6,6a,7,8,9,10,10a-octahydrophenanthridine

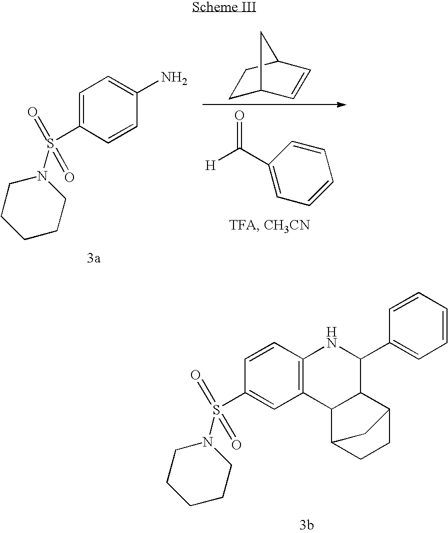

To commercially available 4-(piperidin-1-ylsulfonyl)aniline 3a (0.06 g, 0.25 mmol) in 3 ml acetonitrile was added benzaldehyde (0.026 g, 0.25 mmol), norbornylene (0.035 g, 0.37 mmol), and TFA (Trifluoroacetic acid: 0.028 g, 0.25 mmol). The reaction stirred at room temperature for 18 hours. The desired compound 3b precipitated out of solution as the TFA salt and was collected by filtration to provide a white solid (64.9 mgs). MS: m/z 423.1 (MH+)

### Example 4

### Synthesis of Compound (4d): Bridged N-(6-phenyl-5,6,6a,7,8,9,10,10a-octahydrophenanthridin-2-yl)pentanamide

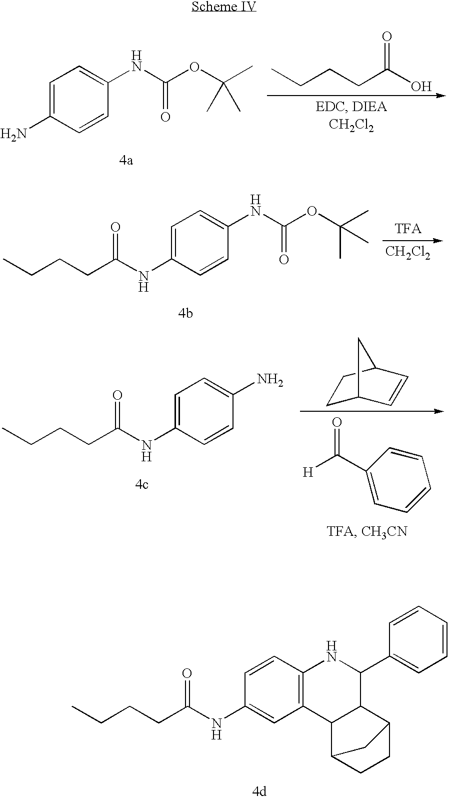

A. Synthesis of compound 4b. To commercially available 4a (0.2 g, 0.96 mmol) in methylene chloride (3 ml) was added commercially available valeric acid (0.096 g, 0.96 mmol), DIEA (N,N-Diisopropylethylamine: 0.25 g, 1.92 mmol), and EDC (1-ethyl-3-(3-dimethylaminopropyl)carbodiimide hydrochloride: 0.18 g, 0.96 mmol). The reaction stirred at room temperature for 18 hours. The organic layer was washed with 1N HCl and dried over sodium sulfate and concentrated. Crude 4b was used in the next step without further purification.

B. Synthesis of compound 4c. To 4b was added 1 ml of methylene chloride and 1 ml of TFA. The reaction stirred at room temperature for 2 hours. The reaction mixture was concentrated. Crude 4c was used in the next step.

C. Synthesis of compound 4d. To 4c (0.1 g, 0.52 mmol) in 3 ml of acetonitrile was added benzaldehyde (0.051 g, 0.48 mmol), norbornylene (0.067 g, 0.72 mmol), and TFA (0.055 g, 0.48 mmol). The reaction mixture stirred for 18 hours at room temperature and was concentrated. The crude mixture was purified using normal phase chromatography (ethyl acetate/hexanes, 10 to 80% gradient) to provide 65.4 mgs of 4d. MS: m/z 375.2 (MH+).

### Example 5

### Synthesis of Compound (5d): Bridged 1-(6-phenyl-5,6,6a,7,8,9,10,10a-octahydrophenanthridin-2-yl)-3-propylurea

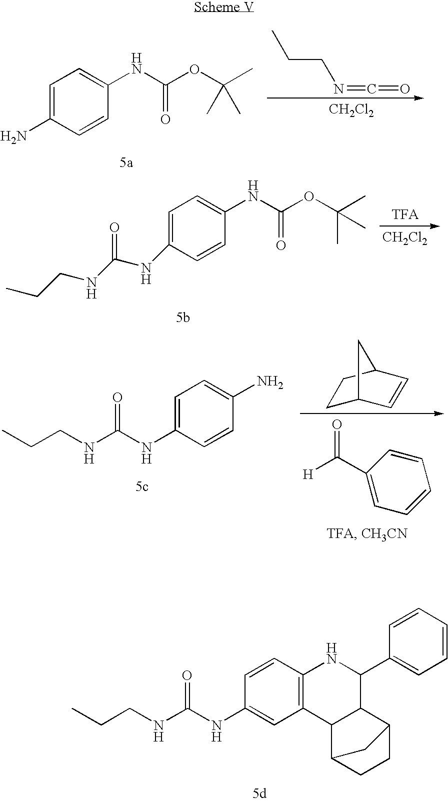

A. Synthesis of compound 5b: To commercially available 5a (0.3 g,. 1.44 mmol) in methylene chloride (4 ml) was added propyl isocyanate (0.11 g, 1.44 mmol). The reaction stirred at room temperature for 18 hours. A white solid precipitated out of solution. The white solid was collected by filtration and determined to be 5b.

B. Synthesis of compound 5c. To 5b was added 1 ml of TFA and 1 ml of methylene chloride. The reaction mixture stirred at room temperature for 2 hours. The mixture was concentrated and used in the next step without further purification.

C. Synthesis of compound 5d: To 5c (0.09 g, 0.46 mmol) in 3 ml of acetonitrile was added benzaldehyde (0.049 g, 0.46 mmol), norbornylene (0.066 g, 0.69 mmol), and TFA (0.052 g, 0.46 mmol). The reaction mixture stirred for 18 hours at room temperature. The desired product precipitated out of solution and was collected by filtration to yield 5d as a white solid and TFA salt (37.5 mgs). MS: m/z 376.2 (MH+).

### Example 6

### Synthesis of compound 6: Bridged 1-propyl-3-(phenyl-5,6,6a,7,8,9,10,10a-octahydrophenanthridin-2-yl)thiourea

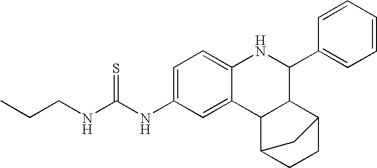

Compound 6 was synthesized in the same manner as compound 5d except that propyl thioisocyanate was used instead of propyl isocyanate. The desired compound was purified by normal phase chromatography as described above. The desired compound was purified by normal phase chromatography as described above. MS: m/z 392.1 (MH+).

### Example 7

### Synthesis of compound 7: Bridged 1-propyl-3-(6-p-tolyl-5,6,6a,7,8,9,10,10a-octahydrophenanthridin-2-yl)urea

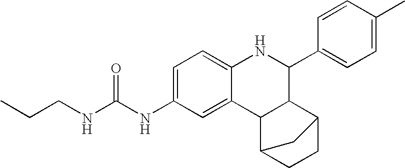

Compound 7 was synthesized in the same manner as compound 5d except that p-tolualdehyde was used instead of benzaldehyde. The desired compound was purified by normal phase chromatography as described above. MS: m/z 390.2 (MH+).

### Example 8

### Synthesis of compound 8: Bridged 1-propyl-3-(6-p-tolyl-5,6,6a,7,8,9,10,10a-octahydrophenanthridin-2-yl)thiourea

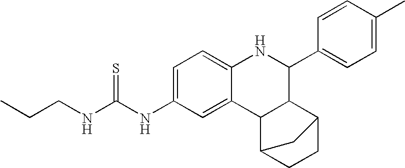

Compound 8 was synthesized in the same manner as compound 6 except that p-tolualdehyde was used instead of benzaldehyde. The desired compound was purified by normal phase chromatography as described above. MS: m/z 406.2 (MH+).

### Example 9

### Synthesis of compound 9: Bridged 6-(2,6-dichlorophenyl)-2-(piperidin-1-ylsulfonyl)-5,6,6a,7,8,9,10,10a-octahydrophenanthridine

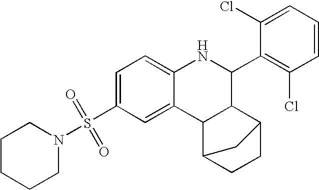

Compound 9 was synthesized in the same manner as compound 3b except that 2,6-dichlorobenzaldehyde was used instead of benzaldehyde. MS: m/z 491.1 (MH+).

### Example 10

### Synthesis of compound 10: Bridged 2-(piperidin-1-ylsulfonyl)-6-p-tolyl-5,6,6a,7,8,9,10,10a-octahydrophenanthridine

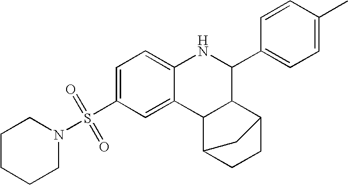

Compound 10 was synthesized in the same manner as compound 3b except that p-tolualdehyde was used instead of benzaldehyde. The desired compound was purified by normal phase chromatography as described above. MS: m/z 437.2 (MH+).

### Example 11

### Synthesis of compound 11: Bridged N,N-dimethyl-6-phenyl-5,6,6a,7,8,9,10,10a-octahydrophenanthridine-2-sulfonamide

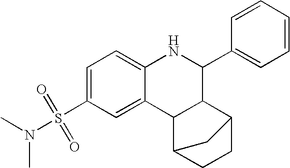

Compound 11 was synthesized in the same manner as compound 3b except that 4-amino-N,N-dimethylbenzenesulfonamide was used instead of 3a. MS: m/z 383.1 (MH+). ¹H NMR (CD₂Cl₂) δ 7.55 (s, 1H), 7.21-7.37 (m, 6H), 6.53-6.60 (d, J=8 Hz, 1H), 4.11-4.19 (br s, 1H), 3.52-3.60 (d, J=10 Hz, 1H), 2.61-2.70 (m, 2H), 2.56 (s, 6H), 2.11-2.20 (m,1H), 2.01-2.04 (m, 1H), 1.10-1.65 (m, 6H).

### Example 12

### Synthesis of compound 12: Bridged 3-methoxy-N-(6-phenyl-5,6,6a,7,8,9,10,10a-octahydrophenanthridin-2-yl)propanamide

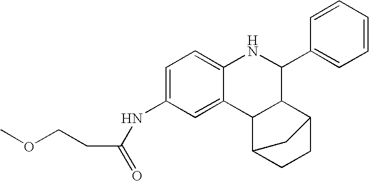

Compound 12 was synthesized in the same manner as compound 4d except that 3-methoxy propionic acid was used instead of valeric acid. MS: m/z 377.3 (MH+).

### Example 13

### Synthesis of compound 13: Bridged N-(6-phenyl-5,6,6a,7,8,9,10,10a-octahydrophenanthridin-2-yl)oxazole-4-carboxamide

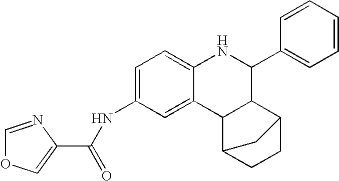

Compound 13 was synthesized in the same manner as compound 4d except that 4-oxazole carboxylic acid was used instead of valeric acid. MS: m/z 386.2 (MH+).

### Example 14

### Synthesis of compound 14: Bridged N-(6-phenyl-5,6,6a,7,8,9,10,10a-octahydrophenanthridin-2-yl)-2-(tetrahydrofuran-2-yl) acetamide

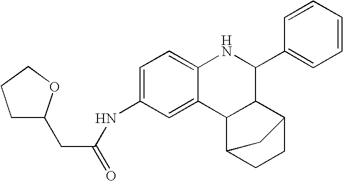

Compound 14 was synthesized in the same manner as compound 4d except that (tetrahydro-furan-2-yl)-acetic acid was used instead of valeric acid. MS: m/z 403.3 (MH+).

### Example 15

### Synthesis of compound 15: Bridged 3-methoxy-N-(6-p-tolyl-5,6,6a,7,8,9,10,10a-octahydrophenanthridin-2-yl)propanamide

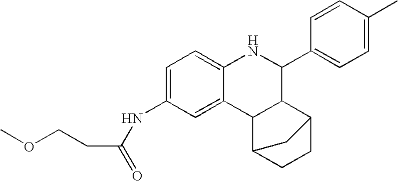

Compound 15 was synthesized in the same manner as compound 12 except that p-tolualdehyde was used instead of benzaldehyde. MS: m/z 391.3 (MH+).

### Example 16

### Synthesis of compound 16: Bridged 2-(tetrahydrofuran-2-yl)-N-(6-p-tolyl-5,6,6a,7,8,9,10,10a-octahydrophenanthridin-2-yl)acetamide

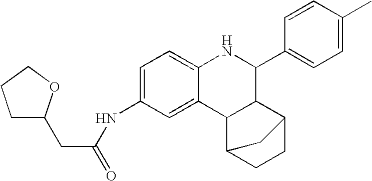

Compound 16 was synthesized in the same manner as compound 14 except that p-tolualdehyde was used instead of benzaldehyde. MS: m/z 417.3 (MH+).

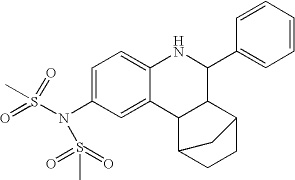

### Example 17

### Synthesis of compound 17: Bridged 4-(6-phenyl-5,6,6a,7,8,9,10,10a-octahydro phenanthridin-2-yl)morpholine

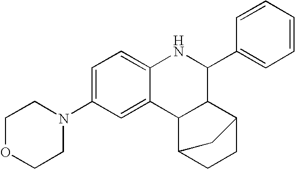

Compound 17 was synthesized in the same manner as compound 3b except that p-morpholinoaniline was used instead of 3a. The desired compound was purified by normal phase chromatography as described above. MS: m/z 361.2 (MH+).

### Example 18

### Synthesis of compound 18: Bridged 4-(6-p-tolyl-5,6,6a,7,8,9,10,10a-octahydro phenanthridin-2-yl)morpholine

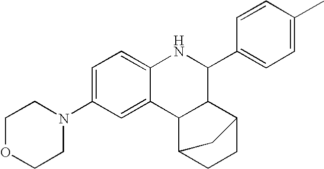

Compound 18 was synthesized in the same manner as compound 17 except that p-tolualdehyde was used instead of benzaldehyde. The desired compound was purified by normal phase chromatography as described above. MS: m/z 375.2 (MH+).

### Example 19

### Synthesis of compound 19: Bridged N-(6-phenyl-5,6,6a,7,8,9,10,10a-octahydrophenanthridin-2-yl)cyclopropanecarboxamide

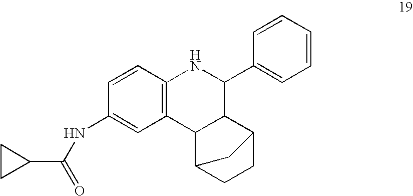

Compound 19 was synthesized in the same manner as compound 4d except that cyclopropyl carboxylic acid was used instead of valeric acid. MS: m/z 359.3 (MH+).

### Example 20

### Synthesis of compound 20: Bridged N-(6-(2,6-dichlorophenyl)-5,6,6a,7,8,9,10,10a-octahydrophenanthridin-2-yl)cyclopropanecarboxamide

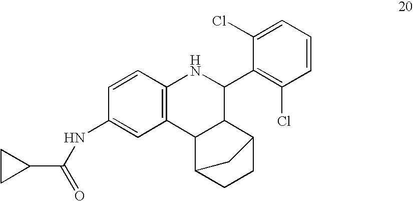

Compound 20 was synthesized in the same manner as 19 except that 2,6-dichlorobenzaldehyde was used instead of benzaldehyde. MS: m/z 427.2 (MH+).

### Example 21

### Synthesis of compound 21: Bridged N-(6-ethyl-5,6,6a,7,8,9,10,10a-octahydrophenanthridin-2-yl)cyclopropanecarboxamide

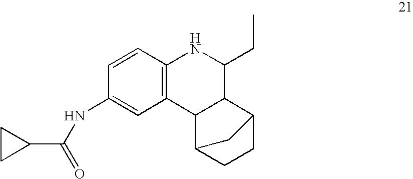

Compound 21 was synthesized in the same manner as compound 19 except that propionaldehyde was used instead of benzaldehyde. MS: m/z 311.3(MH+).

### Example 22

### Synthesis of compound 22: Bridged N-(6-p-tolyl-5,6,6a,7,8,9,10,10a-octahydrophenanthridin-2-yl)cyclopropanecarboxamide

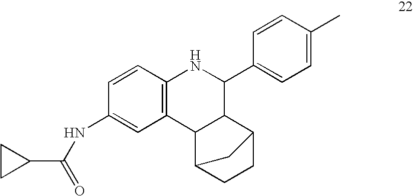

Compound 22 was synthesized in the same manner as compound 19 except that p-tolualdehyde was used instead of benzaldehyde. MS: m/z 373.3(MH+).

### Example 23

### Synthesis of compound 23: Bridged N-(6-phenyl-5,6,6a,7,8,9,10,10a-octahydrophenanthridin-2-yl)acetamide

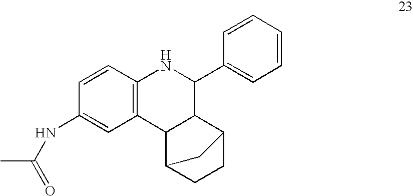

Compound 23 was synthesized in the same manner as 4d except that commercially available 4-amino acetanilide was used instead of 4c. MS: m/z 333.2(MH+).

### Example 24

### Synthesis of compound 24: Bridged N-(6-(2,6-dichlorophenyl)-5,6,6a,7,8,9,10,10a-octahydrophenanthridin-2-yl)acetamide

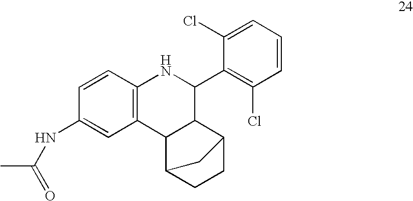

Compound 24 was synthesized in the same manner as 23 except that 2,6-dichlorobenzaldehyde was used instead of benzaldehyde. MS: m/z 401.1(MH+).

### Example 25

### Synthesis of compounds 25a and 25b: Bridged N-(6-p-tolyl-5,6,6a,7,8,9,10,10a-octahydrophenanthridin-2-yl)acetamide

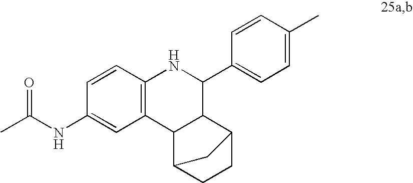

Compounds 25a and 25b were synthesized in the same manner as 23 except the p-tolualdehyde was used instead of benzaldehyde. Upon purification, two compounds were isolated with the same molecular weight and assumed to be diastereomers. MS: m/z 347.3(MH+) and m/z 347.3(MH+).

### Example 26

### Synthesis of compound 26: Bridged 1-(6-(2,6-dichlorophenyl)-5,6,6a,7,8,9,10,10a-octahydrophenanthridin-2-yl)-3-propylurea

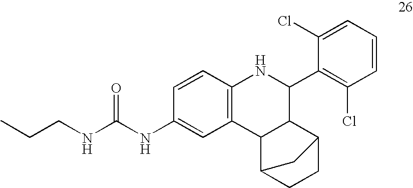

Compound 26 was synthesized in the same manner as 5d except that 2,6-dichlorobenzaldehyde was used instead of benzaldehyde. MS: m/z 444.1 (MH+).

### Example 27

### Synthesis of compound 27: Bridged 1-(6-(2,6-dichlorophenyl)-5,6,6a,7,8,9,10,10a-octahydrophenanthridin-2-yl)-3-propylthiourea

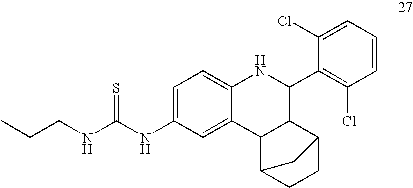

Compound 27 was synthesized in the same manner as compound 6 except that 2,6-dichlorobenzaldehyde was used instead of benzaldehyde. The desired compound was purified by normal phase chromatography as described above. MS: m/z 460.1 (MH+).

### Example 28

### Synthesis of compound 28: Bridged N-benzyl-6-phenyl-5,6,6a,7,8,9,10,10a-octahydrophenanthridine-2-carboxamide

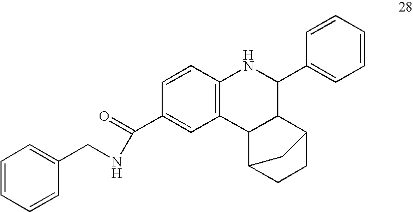

Compound 28 was synthesized in the same manner as compound 2d except that benzylamine was used in place of cyclopropylmethanamine. MS: m/z 409.2 (MH+).

### Example 29

### Synthesis of compound 29: Bridged N-(6-p-tolyl-5,6,6a,7,8,9,10,10a-octahydrophenanthridin-2-yl)pentanamide

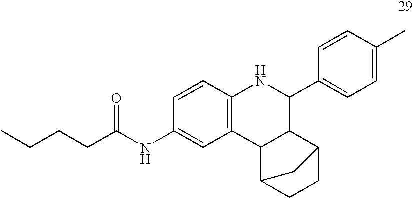

Compound 29 was synthesized in the same manner as compound 4 except that p-tolualdehyde was used instead of benzaldehyde. MS: m/z 389.3 (MH+).

### Example 30

### Synthesis of compound 30: Bridged N,N-dimethyl-6-(pyrimidin-5-yl)-5,6,6a,7,8,9,10,10a-octahydrophenanthridine-2-sulfonamide

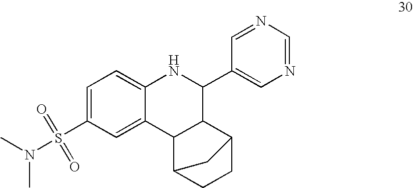

Compound 30 was synthesized in the same manner as compound 11 except that pyrimidine-5-carbaldehyde was used in place of benzaldehyde. MS: m/z 385.1(MH+).

### Example 31

### Synthesis of compound 31: Bridged N,N-dimethyl-6-(thiazol-2-yl)-5,6,6a,7,8,9,10,10a-octahydrophenanthridine-2-sulfonamide:

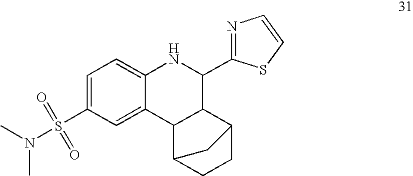

Compound 31 was synthesized in the same manner as compound 11 except that thiazole-2-carbaldehyde was used in place of benzaldehyde. MS: m/z 390.1 (MH+).

### Example 32

### Synthesis of compound 32: Bridged N-(4-(2-(N,N-dimethylsulfamoyl)-5,6,6a,7,8,9,10,10a-octahydrophenanthridin-6-yl)phenyl)acetamide

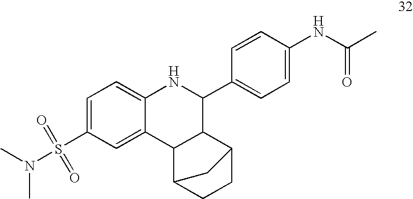

Compound 32 was synthesized in the same manner as compound 11 except that N-(4-formylphenyl)acetamide was used in place of benzaldehyde. MS: m/z 440.2 (MH+).

### Example 33

### Synthesis of compound 33: Bridged N,N-dimethyl-6-(1-methyl-1H-pyrazol-5-yl)-5,6,6a,7,8,9,10,10a-octahydrophenanthridine-2-sulfonamide

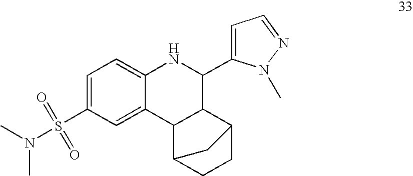

Compound 33 was synthesized in the same manner as compound 11 except that 1-methyl-1H-pyrazole-5-carbaldehyde was used in place of benzaldehyde. MS: m/z 387.2 (MH+).

### Example 34

### Synthesis of compound 34: Bridged 4-(6-phenyl-5,6,6a,7,8,9,10,10a-octahydrophenanthridin-2-ylsulfonyl)morpholine

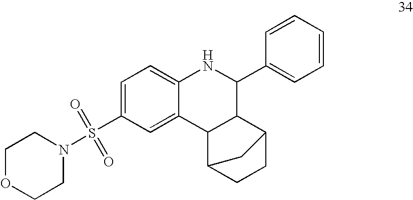

Compound 34 was synthesized in the same manner as compound 3b except that 4-(morpholinosulfonyl)aniline was used in place of 4-(piperidin-1-ylsulfonyl)aniline. MS: m/z 425.2(MH+). ¹H NMR (CD₂Cl₂) δ 7.62 (s, 1H), 7.30-7.45 (m, 6H), 6.65 (d, J=8.8 Hz, 1H), 4.05 (br s, 1H), 3.68-3.77 (m, 4H), 3.62-3.67 (d, J=10 Hz, 1H), 2.91-2.99 (m, 4H), 2.65-2.78 (m, 2H), 2.19-2.29 (t, J=9 Hz, 1H), 2.11-2.15 (m, 1H), 1.10-1.75 (m, 6H).

### Example 35

### Synthesis of compound 35: Bridged 4-(6-(pyrimidin-5-yl)-5,6,6a,7,8,9,10,10a-octahydrophenanthridin-2-ylsulfonyl)morpholine

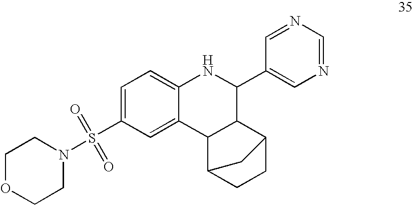

Compound 35 was synthesized in the same manner as compound 3b except that (morpholinosulfonyl)aniline was used in place of 4-(piperidin-1-ylsulfonyl)aniline and pyrimidine-5-carbaldehyde was used in place of benzaldehyde. MS: m/z 427.3 (MH+).

### Example 36

### Synthesis of compound 36: Bridged 4-(6-(thiazol-2-yl)-5,6,6a,7,8,9,10,10a-octahydrophenanthridin-2-ylsulfonyl)morpholine

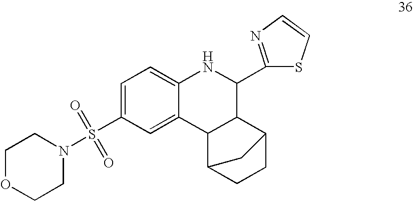

Compound 36 was synthesized in the same manner as compound 3b except that (morpholinosulfonyl)aniline was used in place of 4-(piperidin-1-ylsulfonyl)aniline and thiazole-2-carbaldehyde was used in place of benzaldehyde. MS: m/z 432.2 (MH+). The resulting product was determined to be a 4:1 mixture of 2 diastereomers by ¹H NMR. MS: m/z 432.2 (MH+). ¹H NMR (CD₂Cl₂) δ 7.78-7.79 (d, J=3 Hz, 1H minor), 7.69-7.72 (d, J=3 Hz, 1H major), 7.53-7.58 (m, 1H), 7.41-7.43 (d, J=3 Hz, 1H minor), 7.33-7.38 (m, 1H), 7.31-7.33 (d, J=3 Hz, 1H major), 6.78-6.82 (d. J=8 Hz, 1H minor), 6.71-6.73 (d, J=8 Hz, 1H, major), 5.05 (s, 1H, minor), 4.75 (s, 1H, major), 4.62-4.65 (m, 1H, minor), 4.40-4.42 (m, 1H, major), 3.69-3.73 (m, 4H), 3.13-3.18 (m, 1H, minor), 2.91-2.99 (m, 4H), 2.81-2.85 (m, 1H major), 2.31-2.59 (m, 2H), 1.21-1.66 (m, 5H), 1.11-1.20 (m, 1H major), 0.88-0.94 (m, 1H, minor).

### Example 37

### Synthesis of compound 37: Bridged 4-(6-(1-methyl-1H-pyrazol-5-yl)-5,6,6a,7,8,9,10,10a-octahydrophenanthridin-2-ylsulfonyl)morpholine

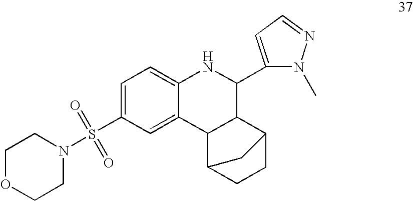

Compound 37 was synthesized in the same manner as compound 3b except that (morpholinosulfonyl)aniline was used in place of 4-(piperidin-1-ylsulfonyl)aniline and 1-methyl-1H-pyrazole-5-carbaldehyde was used in place of benzaldehyde. MS: m/z 429.2 (MH+). The resulting product was determined to be a 3:1 mixture of 2 diastereomers by ¹H NMR. MS: m/z 429.2 (MH+).¹H NMR (CD₂Cl₂) δ 7.61 (s, 1H, major), 7.55 (s, 1H, minor), 7.43 (s, 1H, minor), 7.31-7.36 (m, 2H), 6.67-6.70 (m, 1H), 6.36 (s, 1H, minor), 6.14 (s, 1H, major), 4.41-4.43 (d, 1H, minor), 4.22-4.24 (br s, 1H), 3.99-4.12 (d, J=8 Hz, 1H, major), 3.91 (s, 3H, major), 3.84 (s, 3H, minor), 3.69-3.73 (m, 4H), 3.08-3.11 (m, 1H, minor), 2.91-2.97 (m, 4H), 2.78-2.81 (m, 1H, major), 2.61-2.65 (m, 1H), 2.05-2.37 (m, 3H), 1.11-1.79 (m, 5H), 0.90-0.99 (m, 1H, minor).

### Example 38

### Synthesis of compound 38: Bridged N-cyclohexyl-N-methyl-6-phenyl-5,6,6a,7,8,9,10,10a-octahydrophenanthridine-2-sulfonamide

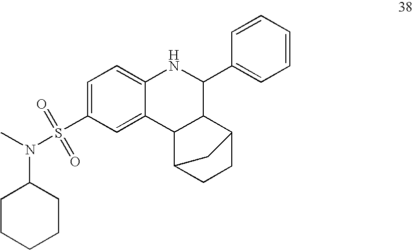

Compound 38 was synthesized in the same manner as compound 3b except that 4-amino-N-cyclohexyl-N-methylbenzenesulfonamide was used in place of 4-(piperidin-1-ylsulfonyl)aniline. MS: m/z 451.2 (MH+).

### Example 39

### Synthesis of compound 39: Bridged 6-phenyl-5,6,6a,7,8,9, 10,10a-octahydrophenanthridine-2-sulfonamide

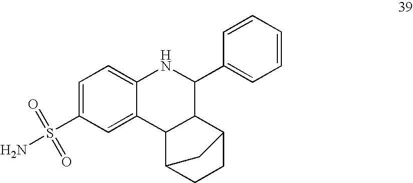

Compound 39 was synthesized in the same manner as compound 3b except that 4-amino-N,N-dimethylbenzenesulfonamide was used in place of 4-(piperidin-1-ylsulfonyl)aniline. MS: m/z 355.1 (MH+).

### Example 40

### Synthesis of compound 40: Bridged 6-(1-methyl-1H-pyrazol-5-yl)-5,6,6a,7,8,9,10,10a-octahydrophenanthridine-2-sulfonamide

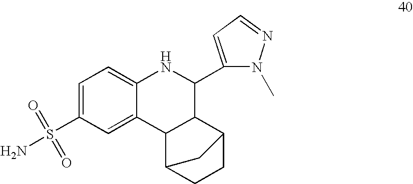

Compound 40 was synthesized in the same manner as compound 3b except that 4-aminobenzenesulfonamide was used in place of 4-(piperidin-1-ylsulfonyl)aniline and 1-methyl-1H-pyrazole-5-carbaldehyde was used in place of benzaldehyde. MS: m/z 359.2 (MH+).

### Example 41

### Synthesis of compound 41: Bridged 6-phenyl-2-(pyrrolidin-1-ylsulfonyl)-5,6,6a,7,8,9,10,10a-octahydrophenanthridine

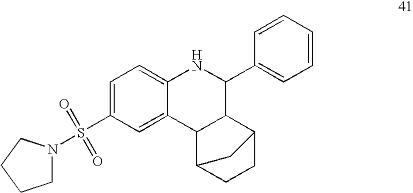

Compound 41 was synthesized in the same manner as compound 3b except that 4-(pyrrolidin-1-ylsulfonyl)aniline was used in place of 4-(piperidin-1-ylsulfonyl)aniline. MS: m/z 409.2 (MH+).

### Example 42

### Synthesis of compound 42: Bridged 6-(1-methyl-1H-pyrazol-5-yl)-2-(pyrrolidin-1-ylsulfonyl)-5,6,6a,7,8,9,10,10a-octahydrophenanthridine

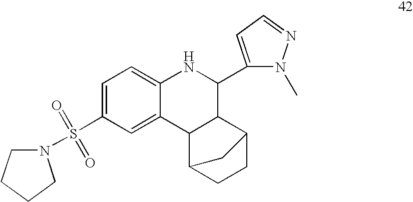

Compound 42 was synthesized in the same manner as compound 3b except that 4-(pyrrolidin-1-ylsulfonyl)aniline was used in place of 4-(piperidin-1-ylsulfonyl)aniline and 1-methyl-1H-pyrazole-5-carbaldehyde was used in place of benzaldehyde. MS: m/z 413.2 (MH+).

### Example 43

### Synthesis of compound 43: Bridged 2-methyl-4-(6-phenyl-5,6,6a,7,8,9,10,10a-octahydrophenanthridin-2-yl)thiazole

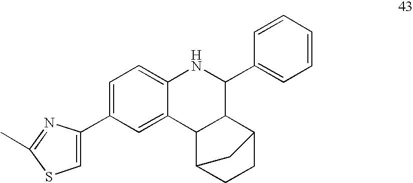

Compound 43 was synthesized in the same manner as compound 3b except that 4-(2-methylthiazol-4-yl)aniline was used in place of 4-(piperidin-1-ylsulfonyl)aniline. MS: m/z 373.3(MH+).

### Example 44

### Synthesis of compound 44: Bridged 2-methyl-4-(6-(1-methyl-1H-pyrazol-5-yl)-5,6,6a,7,8,9,10,1 0a-octahydrophenanthridin-2-yl)thiazole

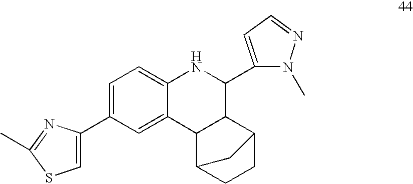

Compound 44 was synthesized in the same manner as compound 3b except that 4-(2-methylthiazol-4-yl)aniline was used in place of 4-(piperidin-1-ylsulfonyl)aniline and 1-methyl-1H-pyrazole-5-carbaldehyde was used in place of benzaldehyde. MS: M/z 377.3 (MH+).

### Example 45

### Synthesis of compound 45: Bridged 2-butyl-5-(6-phenyl-5,6,6a,7,8,9,10,10a-octahydrophenanthridin-2-yl)-1,3,4-oxadiazole

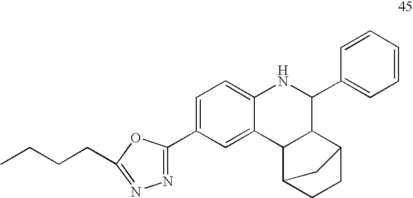

Compound 45 was synthesized in the same manner as compound 3b except that 4-(5-butyl-1,3,4-oxadiazol-2-yl)aniline was used in place of 4-(piperidin-1-ylsulfonyl)aniline. MS: m/z 400.4(MH+).

### Example 46

### Synthesis of compound 46: Bridged N-(6-phenyl-5,6,6a,7,8,9,10,10a-octahydrophenanthridin-2-ylsulfonyl)acetamide

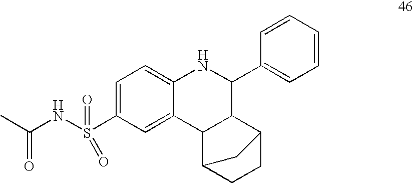

Compound 46 was synthesized in the same manner as compound 3b except that N-(4-aminophenylsulfonyl)acetamide was used in place of 4-(piperidin-1-ylsulfonyl)aniline. MS: m/z 397.3 (MH+).

### Example 47

### Synthesis of compound 47: Bridged N-(6-(1-methyl-1H-pyrazol-5-yl)-5,6,6a,7,8,9,10,10a-octahydrophenanthridin-2-ylsulfonyl)acetamide

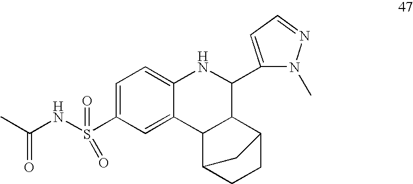

Compound 47 was synthesized in the same manner as compound 3b except that N-(4-aminophenylsulfonyl)acetamide was used in place of 4-(piperidin-1-ylsulfonyl)aniline and 1-methyl-1H-pyrazole-5-carbaldehyde was used in place of benzaldehyde. MS: m/z 401.3(MH+).

### Example 48

### Synthesis of compound 48: 4-phenyl-8-(piperidin-1-ylsulfonyl)-3a,4,5,9b-tetrahydro-1H-cyclopenta[c]quinoline

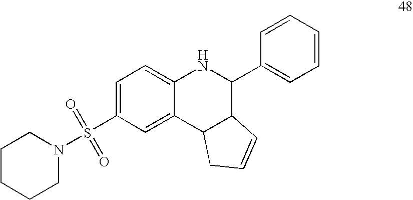

Compound 48 was synthesized in the same manner as 3b except that cyclopentadiene was used in place of norbornylene. MS: m/z 395.2(MH+).

### Example 49

### Synthesis of compound 49: N,N-dimethyl-4-phenyl-3a,4,5,9b-tetrahydro-1H-cyclopenta[c]quinoline-8-sulfonamide

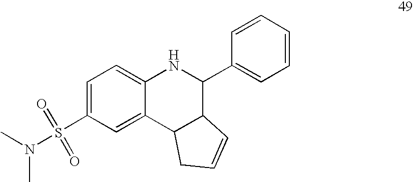

Compound 49 was synthesized in the same manner as 3b except that cyclopentadiene was used in place of norbornylene and 4-amino-N,N-dimethylbenzenesulfonamide was used in place of 4-(piperidin-1-ylsulfonyl)aniline. MS: m/z 355.1 (MH+).

### Example 50

### Synthesis of compound 50: Bridged 5-(isopropylsulfonyl)-N,N-dimethyl-6-phenyl-5,6,6a,7,8,9,10,10a-octahydrophenanthridine-2-sulfonamide

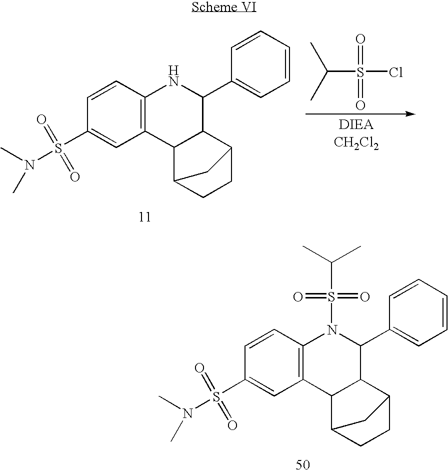

To compound 11 (0.05 g, 0.131 mmol) in methylene chloride (2 mL) was added DIEA (0.034 g, 0.261 mmol) and propane-2-sulfonyl chloride (0.018 g, 0.131 mmol). The reaction mixture stirred at room temperature for 16 hours. The organics were washed with water, dried over sodium sulfate and concentrated. The crude mixture was purified using reverse chromatography to yield 1.8 mgs of 50. MS: m/z 489.1 (MH+).

### Example 51

### Synthesis of compound 51: Bridged N-(6-phenyl-5,6,6a,7,8,9,10,10a-octahydrophenanthridin-2-yl)propane-2-sulfonamide

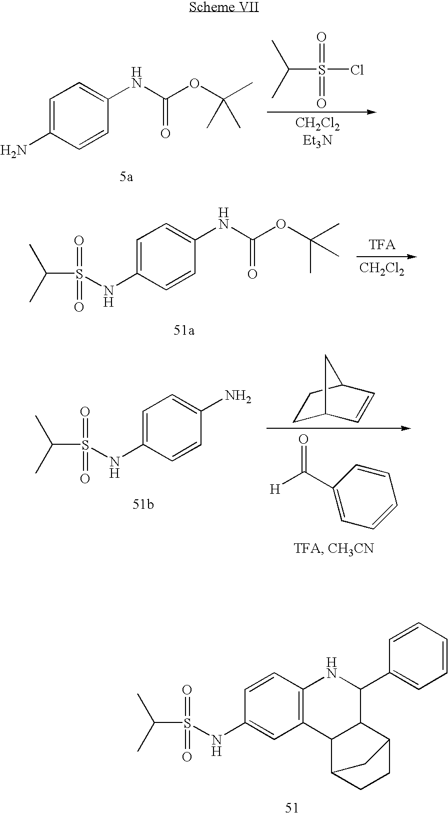

A. Synthesis of 51a. To tert-butyl 4-aminophenylcarbamate 5a (0.1 g, 0.48 mmol) in methylene chloride was added triethylamine (0.13 mL, 0.96 mmol) and propane-2-sulfonyl chloride (0.068 g, 0.48 mmol). The reaction mixture stirred for 20 hours at room temperature. The organics were washed with ammonium chloride solution, extracted with methylene chloride, dried over sodium sulfate and concentrated. The crude material was used in the next step without further purification.

B. Synthesis of 51b. To 51a was added 1 mL of methylene chloride and 1 mL of TFA. The reaction mixture stirred for 16 hours at room temperature. The reaction mixture was concentrated and the crude material was used in the next step without further purification.

C. Synthesis of 51. To 51b (0.1 g, 0.48 mmol) in 3 ml of acetonitrile was added benzaldehyde (0.051 g, 0.48 mmol), norbornylene (0.067 g, 0.72 mmol), and TFA (0.055 g, 0.48 mmol). The reaction mixture stirred for 18 hours at room temperature and was concentrated. The crude mixture was purified using normal phase chromatography using an ISCO and 10 to 80% gradient (ethyl acetate/hexanes) to provide 65.4 mgs of 51. MS: m/z 397.2(MH+).

### Example 52

### Synthesis of compound 52: Bridged N-(methylsulfonyl)-N-(6-phenyl-5,6,6a,7,8,9,10,10a-octahydrophenanthridin-2-yl)methanesulfonamide

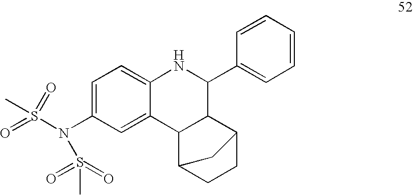

Compound 52 was synthesized in the same manner as compound 51 except that methanesulfonyl chloride was used in place of propane-2-sulfonyl chloride. The reaction with methanesulfonyl chloride yielded the di-alkylated amine rather than the mono-alkylated amine. MS: m/z 447.1(MH+).

### Example 53

### Screening Methods

The ability of compounds to act as agonists or inverse agonists at human CB2 and CB1 receptors (hCB2, hCB1, respectively) was determined by measuring changes in intracellular cAMP levels. Chinese Hamster Ovary (CHO-K1) cell lines stably expressing hCB2 (Genebank: X74328) or hCB1 (Genebank: X54937) were purchased from Euroscreen (Gosselies, Belgium).

Cell lines were grown in suspension in EX-CELL 302 CHO Serum-free medium (Sigma, catalog #14324C) supplemented with 1% Fetal Bovine Serum, glutamine and with or without non-essential amino-acids under 0.4 mg/mL G418 selection.

Receptor mediated responses were determined by measuring changes in intracellular cAMP using LANCE cAMP detection kit (catalog #AD0264, PerkinElmer, Wellesley, Mass.) based on time-resolved fluorescence resonance energy transfer (TR-FRET). Changes in cAMP were determined in cells pre-incubated with IBMX (isobutyl methylxanthine) and prestimulated with NKH-477 (a water soluble forskolin derivative, catalog #1603, Tocris, Ellisville, Mo.) to increase basal cAMP levels as detailed below.

On the day of the experiment, cells were spun at low speed for 5 min at room temperature. The supernatant was removed and cells were resuspended in stimulation buffer (Hanks Buffered Salt Solution/5 mM HEPES, containing 0.5 mM IBMX (catalog #17018, Sigma) and 0.02% BSA (PerkinElmer, catalog #CR84-100)). Cell clumps were removed by filtering through cell strainer 40 μm (BD Falcon, Discovery Labware, Bedford, Mass.) and diluted to 2×10⁵ cells/mL. Antibody supplied with the LANCE cAMP immunoassay kit was then added according to the manufacturer's instructions. An aliquot of cells was taken for un-induced controls. To the remaining cells was added NKH-477 (a water soluble forskolin derivative, Tocris catalog #1603) to a final concentration of 2-8 μM. Cells were then incubated for either 25 or 30 min at room temperature prior to adding to Proxiplates containing test compounds (final DMSO concentration was less than 0.5%) with a Multidrop bulk dispenser, followed by a sixty minute incubation at room temperature. The response was stopped by addition of the detection mix supplied with the LANCE kit.

The reagents were allowed to equilibrate for three hours prior to reading on an Envision multi-mode detector (PerkinElmer). TR-FRET was measured using a 330-380 nm excitation filter, 615 and 665 nm emission filters, dichroic mirror 380 nm and Z=1 mm.

Cyclic AMP concentrations in each well were back-calculated from a cAMP standard curve run concurrently during each assay. Each plate contained 16 wells of forskolin stimulated cells and 16 wells of forskolin plus CP55,940-treated. CP55,940-treated cells were treated with CP55,940 (Tocris catalog #0949) at 1 μM and the maximal response was used as the full range (100%) standard. WIN55,212 (Tocris catalog #1038) was used as an internal standard. Concentrations of cAMP were expressed as a percent of the difference of these two groups of wells. Concentration-response data including EC₅₀ (the concentration of compound producing 50% of the maximal response) and intrinsic activity (the percent maximal activation compared to full activation by CP55,940) were determined using a four-parameter non-linear regression algorithm (Xlfit equation 251, IDBS). Assays were performed with at least three replicates per compound and averaged results for the several compounds of the invention are shown in Table I.

TABLE I

| | hCB2 EC50 | hCB2 % | hCB1 EC50 | hCB1 |
|---|---|---|---|---|
| Compound | (nM) | Max Range | (nM) | % Max Range |
| (2d) | 19.0 | 84 | >10,000 | N/A |
| (3b) | 0.38 | 101 | >10,000 | N/A |
| (4d) | 6.9 | 92 | >10,000 | N/A |
| (5d) | 289 | 73 | 2149 | 46 |
| (6) | 99.8 | 76 | 1861 | 53 |
| (7) | >10,000 | N/A | >10,000 | N/A |
| (8) | >10,000 | N/A | 2045 | 63 |
| (9) | 79.4 | 75 | >10,000 | N/A |
| (10) | 21.0 | 98 | >10,000 | N/A |
| (11) | 0.98 | 99 | >10,000 | N/A |
| (12) | 12.4 | 82 | >10,000 | N/A |
| (13) | 680 | 57 | >10,000 | N/A |
| (14) | 9.1 | 92 | >10,000 | N/A |
| (15) | >10,000 | N/A | 2359 | 56 |
| (16) | 102.4 | 52 | >10,000 | N/A |
| (17) | 140 | 94 | >10,000 | N/A |
| (18) | >10,000 | N/A | >10,000 | N/A |
| (19) | 187 | 49 | 1701 | 53 |
| (20) | >10,000 | N/A | >10,000 | N/A |
| (21) | 1195 | 84 | >10,000 | N/A |
| (22) | 14 | 53 | 1815 | 115 |
| (23) | 43 | 82 | 2605 | 88 |
| (24) | >10,000 | N/A | >10,000 | N/A |
| (25a) | >10,000 | N/A | 3277 | 78 |
| (25b) | >10,000 | N/A | 3537 | 107 |
| (26) | >10,000 | N/A | >10,000 | N/A |
| (27) | >10,000 | N/A | >10,000 | N/A |
| (28) | 372.6 | 84.4 | >10,000 | N/A |
| (29) | 66.7 | 54.8 | >10,000 | N/A |
| (30) | 72.8 | 99.3 | >10,000 | N/A |
| (31) | 102.9 | 89.2 | >10,000 | N/A |
| (32) | 225 | 81.3 | >10,000 | N/A |
| (33) | 7.5 | 94.9 | >10,000 | N/A |
| (34) | 0.56 | 101.8 | >10,000 | N/A |
| (35) | 1302 | 66.6 | >10,000 | N/A |
| (36) | 4116 | 57.1 | >10,000 | N/A |
| (37) | 1.7 | 92.6 | >10,000 | N/A |
| (38) | 8.5 | 79.5 | >10,000 | N/A |
| (39) | 17.7 | 83.3 | 3037 | 172 |
| (40) | 2004 | 82.7 | >10,000 | N/A |
| (41) | 0.81 | 103.6 | >10,000 | N/A |
| (42) | 0.91 | 100.3 | >10,000 | N/A |
| (43) | 217 | 64.6 | 3846 | 182 |
| (44) | 187 | 70.2 | 2930 | 111 |
| (45) | 70.4 | 96.8 | >10,000 | N/A |
| (46) | 36.7 | 96.9 | >10,000 | N/A |
| (47) | 1486 | 82.5 | >10,000 | N/A |
| (48) | 73.9 | 82.3 | >10,000 | N/A |
| (49) | >10,000 | N/A | 1037 | 61.8 |
| (50) | 1501 | 81.2 | >10,000 | N/A |
| (51) | 1.62 | 96.8 | 2212 | 100 |
| (52) | 0.63 | 101.6 | >10,000 | N/A |

N/A: Response <40% assay range; Activity on hCB1 represents inverse agonism.

### Example 54

### Acetic acid-induced writhing assay in mice

This test identifies compounds which exhibit analgesic activity against visceral pain or pain associated with activation of low pH-sensitive nociceptors [see Barber and Gottschlich (1986) Med. Res. Rev. 12: 525-562; Ramabadran and Bansinath (1986) Pharm. Res. 3: 263-270]. Intraperitoneal administration of dilute acetic acid solution causes a writhing behavior in mice. A writhe is defined as a contraction of the abdominal muscles accompanied by an extension of the forelimbs and elongation of the body. The number of writhes observed in the presence and absence of test compounds is counted to determine the analgesic activity of the compounds.

Male ICR mice, 20-40 grams in weight, served as experimental subjects. To determine the activity and potency of test compounds, mice were weighed and then injected with different doses of the compound solution or vehicle subcutaneously in the back of the neck and then returned to their home cages. Thirty minutes later, body temperatures were obtained using a digital rectal probe and then mice were immediately injected with 10 ml/kg of a 0.6% (v/v) acetic acid solution into the right lower quadrant of the abdomen. Mice were then placed in individual observation chambers (usually a 4000 ml beaker) with a fine layer of bedding at the bottom and the recording of the number of writhes begun immediately. The number of writhes was counted over a 15-min period starting from the time of the acetic acid injection. Raw data were analyzed using a one-way ANOVA followed by Dunnett's post-tests. For dose-response analysis, raw data were converted to % maximum possible effect (% MPE) using the formula:

% MPE=((W_{c}−Wᵥ)/(0−Wᵥ))*100

where Wc is the number of writhes in compound-treated mice and Wv is the mean number of writhes in vehicle-treated mice. The dose which elicited 50% attenuation of hypersensitivity (ED₅₀) was determined using linear regression analysis. (Tallarida & Murray, 1987).

Results in the mouse acetic acid-induced writhing assay for compounds 3b, 34 and 46 are shown in Table II.

TABLE II

| COMPOUND | DOSE (mg/kg) | % MPE | Δ BODY TEMP. (° C.) |
|---|---|---|---|
| (3b) | 10 | 12 | 0.4 |
| (34) | 10 | 23 | 0.1 |
| (46) | 10 | 41 | 0.2 |

Δ BODY TEMP.: observed reduction in body temperature

We claim:

1. A compound having the structure of formula I or a pharmaceutically acceptable salt, ester, solvate, stereoisomer, or racemate thereof; 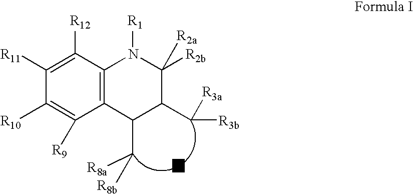
wherein
B is a bond or a 1, 2, 3, or 4 carbon atom chain which completes a 4-8 membered saturated, singly unsaturated, or doubly unsaturated carbocyclic ring, wherein each of the ring atoms in B are optionally substituted and each of said ring substitutents is independently selected from the group consisting of H, C₁-C₄ alkyl, C₁-C₄ alkoxy, C₁-C₄ halo alkyl, C₁-C₄ haloalkoxy, fluoro, chloro, cyano, hydroxyl, C₁-C₄ hydroxy alkyl, or, when B completes a seven or eight membered ring, two carbon atoms in the chain B which are separated by at least one carbon atom in the chain B are optionally bridged by a methylene or an ethylene moiety;
R₁ is selected from the group consisting of H, C₁-C₅ straight, branched or cyclic alkyl, —COR₁₃, —SO₂R₁₃, benzyl, alkyl or halo substituted benzyl, where R₁₃ is —OH, C₁-C₄ straight or branched alkyl;
R₂ₐ is -L-R₁₄ where L is —Xₜ(CO)ₘXₚ(CH₂)ₙY_{q}— and X is O or NH, Y is O or S, m is 0 or 1, n is 0, 1, or 2, p is 0 or 1, q is 0 or 1, t is 0 or 1 provided that where p and t are both 1, then m=1, q is zero and X is NH; wherein R₁₄ is selected from the group consisting of optionally substituted aryl, optionally substituted heteroaryl, optionally substituted cycloalkyl, optionally substituted cycloheteroalkyl, optionally substituted straight or branched C₁-C₈ alkyl, or optionally substituted straight or branched C₁-C₈ heteroalkyl, wherein, when R₁₄ comprises aryl or heteroaryl, said aryl or heteroaryl moiety is either a single 5 or 6 membered ring or a fused bicyclic moiety;
R_{2b} is H or methyl;
R₃ₐ and R₈ₐ are
(i) taken together to form a methylene or ethylene bridge wherein the bridge carbons of said bridge are unsubstituted or substituted such that said bridge carbon substitutents are each independently selected from the group consisting of methyl, chloro, fluoro;
(ii) selected such that one is H and the other forms a methylene or ethylene bridge to a carbon atom in the chain B; or
(iii) each independently selected from the group consisting of a bond forming a second ring bond with an adjacent ring carbon atom, H, C₁-C₄ alkyl, C₁-C₄ alkoxy, C₁-C4 halo alkyl, C₁-C4 haloalkoxy, fluoro, chloro, cyano hydroxyl, C₁-C₄ hydroxy alkyl;
R_{3b} and R_{8b} are each independently selected from the group consisting of H and methyl;
R₉, R₁₁, and R₁₂ are each independently selected from the group consisting of H, halo, nitro, cyano, hydroxyl, amino, C₁₋₄ alkyl, C₁₋₄ alkoxy, C₁₋₄ haloalkoxy, C₁₋₄ haloalkyl, perhalomethoxy, perhalomethyl, and cyclopropyl;
R₁₀ is selected from the group consisting of —(CH₂)ᵣ—CO—(CH₂)—R₁₅, —(CH₂)ᵣ—COO—(CH₂)ᵣ—R₁₅, —(CH₂)ᵣ—CS—(CH₂)ᵣ—R₁₅, —(CH₂)ᵣN(R₁₆)((CH₂)ᵣ—R₁₇), —(CH₂)ᵣ—CO—N(R₁₆)((CH₂)ᵣR₁₇), —(CH₂)ᵣNH—CO—(CH₂)ᵣR₁₅, —(CH₂)ᵣ—CS—N(R₁₆)((CH₂)ᵣR₁₇), —(CH₂)ᵣNH—CS—(CH₂)ᵣR₁₅, —(CH₂)ᵣSO₂—(CH₂)ᵣR₁₅, —(CH₂)ᵣSO—(CH₂)ᵣR₁₅, —(CH₂)ᵣS—(CH₂)ᵣR₁₅, —(CH₂)ᵣSO₂—N(R₁₆)((CH₂)ᵣ—R₁₇), —(CH₂)ᵣNH—SO₂—(CH₂)ᵣR₁₅, —(CH₂)ᵣNH—CO—N(R₁₆)((CH₂)ᵣR₁₇), —(CH₂)ᵣNH—CS—N(R₁₆)((CH₂)ᵣR₁₇), and an optional alkylene linker coupled to a 5 or 6-membered heteroaryl ring optionally substituted with R₁₅; where each r is independently selected from the group of 0, 1, 2, and 3; where R₁₅ is selected from the group consisting of H, optionally substituted straight or branched C₁-C₈ alkyl, optionally substituted straight or branched C₁-C₈ heteroalkyl, optionally substituted C₃-C₈ cycloalkyl, optionally substituted 5, 6, or 7-membered cycloheteroalkyl, optionally substituted aryl, optionally substituted 5 or 6-membered or bicyclic heteroaryl; R₁₆ and R₁₇ are either taken together to form an optionally substituted cycloheteroalkyl or are each independently selected from the group consisting of substituted straight or branched C₁-C₈ alkyl, optionally substituted straight or branched C₁-C₈ heteroalkyl, optionally substituted C₃-C₈ cycloalkyl, optionally substituted 5, 6, or 7-membered cycloheteroalkyl, optionally substituted aryl, optionally substituted 5 or 6-membered or bicyclic heteroaryl;
provided that when R₂ₐ is unsubstituted phenyl, R₁, R_{2b}, R_{3b}, R_{8b}, R₉, R₁₁, R₁₂ are all H, R₃ₐ,
R₃ₐ, R₈ₐ and B taken together complete an unsubstituted norbornanyl moiety, and
R₁₀ is —CO—N R₁₆R₁₇, then R₁₆ is —(CH₂)ₛ—R₁₈ with R₁₈ is optionally substituted
aryl or cyclopropyl and s is 1, 2, or 3 and R₁₇ is H; provided further that when R₁₀ is —COOH, R₁, R_{2b}, R_{3b}, R_{8b}, R₉, R₁₁, R₁₂ are all H, R₃ₐ,
R₈ₐ and B taken together complete an unsubstituted norbornanyl moiety, and m, n, p, q, and t are all zero, then R₁₄ is not phenyl.

2. A compound according to claim 1, wherein R₃ₐ, R₈ₐ and B taken together complete an optionally substituted, bridged six- or seven-membered ring.

3. A compound according to claim 2, wherein said bridge is a methylene bridge.

4. A compound according to claim 3, wherein R₃ₐ, R₈ₐ and B taken together complete an optionally substituted norbornanyl moiety.

5. A compound according to claim 1 wherein R₃ₐand R₈ₐ are each H, and B completes a cyclopentenyl moiety.

6. A compound according to claim 1, wherein R₁ is H.

7. A compound according to claim 1, wherein R₁ is C₁-C₅ alkyl.

8. A compound according to claim 1, wherein R₉, R₁₁, and R₁₂ is H.

9. A compound according to claim 1, wherein m, p, q, and t are each zero.

10. A compound according to claim 9, wherein n is zero.

11. A compound according to claim 1, wherein R₁₄ is optionally substituted aryl or optionally substituted heteroaryl.

12. A compound according to claim 11, wherein R₁₄ is optionally substituted aryl.

13. A compound according to claim 12, wherein R₁₄ is optionally substituted phenyl.

14. A compound according to claim 11, wherein R₁₄ is optionally substituted heteroaryl.

15. A compound according to claim 14, wherein R₁₄ is optionally substituted 5- or 6-membered heteroaryl.

16. The compound according to claim 1, wherein R₁₄ is optionally substituted straight or branched C₁-C₆ alkyl.

17. A compound according to claim 1, wherein each r is zero.

18. A compound having the structure of formula II: 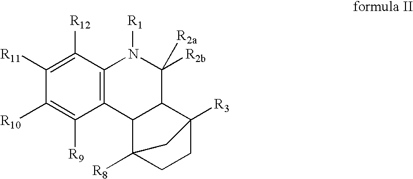
or a pharmaceutically acceptable salt, ester, solvate, stereoisomer, or racemate thereof;
wherein
R₁ is selected from the group consisting of H, C₁-C₅ straight, branched or cyclic alkyl, —COR₁₃, —SO₂R₁₃, wherein R₁₃ is C₁-C₃ straight or branched alkyl;
R₂ₐ is -L-R₁₄ where L is —(NH)ₚ(CH₂)ᵣY_{q}— and Y is C═O or C═S, p and q are each independently 0 or 1; wherein R₁₄ is selected from the group consisting of optionally substituted aryl, optionally substituted cycloalkyl, optionally substituted heterocyclyl, optionally substituted straight or branched C₁-C₄ alkyl, or optionally substituted straight or branched C₁-C₄ heteroalkyl, wherein, when R₁₄ comprises aryl or heterocyclyl, said aryl or heterocyclyl moiety is either a single 5- or 6-membered ring or a fused bicyclic moiety;
R_{2b} is H or methyl;
R₃ and R₈ are each independently selected from the group consisting of H and C₁-C₃ alkyl;
R₉, R₁₁ and R₁₂ are each independently selected from the group consisting of H, halo, cyano, hydroxyl, amino, and C₁₋₄ alkyl;
R₁₀ is selected from the group consisting of —(CH₂)—COO—(CH₂)ᵣ—R₁₅, —(CH₂)ᵣN(R₁₆)((CH₂)ᵣ—R₁₇), —(CH₂)ᵣR₁₆—(CH₂)ᵣR₁₇, —(CH₂)ᵣ—CO—N(R₁₆)((CH₂)ᵣ—R₁₇), —(CH₂)ᵣNH—CO—(CH₂)ᵣ—R₁₅, —(CH₂)ᵣSO₂—(CH₂)ᵣ—R₁₅, —(CH₂)ᵣSO—(CH₂)ᵣ—R₁₅, —(CH₂)ᵣSO₂—N(R₁₆)((CH₂)ᵣ—R₁₇), —(CH₂)ᵣSO₂—NR₁₆—CO—((CH₂)ᵣ—R₁₇), —(CH₂)ᵣNH—SO₂—(CH₂)ᵣ—R₁₅, —(CH₂)ᵣNH—CO—N(R₁₆)((CH₂)ᵣ—R₁₇), —(CH₂)ᵣNH—CS—N(R₁₆)((CH₂)ᵣ—R₁₇), —(CH₂)ᵣN—(SO₂R₁₆)(SO₂R₁₇), —CO—NH—(CH₂)ᵣ—(C₃—C₆ cycloalkyl) and —CO—NH—(CH₂)ᵣ-(aryl);
wherein each r is independently selected from the group consisting of 0, 1, 2, and 3; where R₁₅ is selected from the group consisting of H, optionally substituted straight or branched C₁-C₆ alkyl, optionally substituted straight or branched C₁-C₆ heteroalkyl, optionally substituted C₃-C₈ cycloalkyl, optionally substituted 5-7 membered heterocyclyl, optionally substituted aryl and optionally substituted 5-6 membered or bicyclic heteroaryl; R₁₆ and R₁₇ are either taken together to form an optionally substituted heterocyclyl or are each independently selected from the group consisting of —H, substituted straight or branched C₁-C₆ alkyl, optionally substituted straight or branched C₁-C₆ heteroalkyl, optionally substituted C₃-C₈ cycloalkyl, optionally substituted 5, 6 or 7-membered heterocyclyl, optionally substituted aryl, optionally substituted 5 or 6-membered heteroaryl;
provided that R₁₀ is not —COOH; and
provided further that when R₂ₐ is unsubstituted phenyl, R₁, R_{2b}, R_{3b}, R_{8b}, R₉, R₁₁ and R₁₂ are all H, and R₁₀ is —CO—NHR₁₆, then R₁₆ is —(CH₂)ₛR₁₈, wherein R₁₈ is optionally substituted aryl or cyclopropyl and s is 1, 2, or 3.

19. A compound of claim 18, wherein, R₁ is H or —SO₂R₁₃:
R_{2b}, R₃, R₈, R₉, R₁₁ and R₁₂ are each H;
R₁₀ is selected from the group consisting of —(CH₂)ᵣ—COO—(CH₂)ᵣ—R₁₅, —(CH₂)ᵣN(R₁₆)((CH₂)ᵣ—R₁₇), —(CH₂)ᵣR₁₆—(CH₂)ᵣ—-R₁₇, —(CH₂)ᵣ—CO—N(R₁₆)((CH₂)ᵣ—R₁₇), —(CH₂)ᵣNH—CO—(CH₂)ᵣ—R₁₅, —(CH₂)ᵣSO₂—(CH₂)ᵣ—R₁₅, —(CH₂)ᵣSO₂—N(R₁₆)((CH₂)ᵣ—R₁₇), —(CH₂)ᵣSO₂—N(R₁₆)—CO—((CH₂)ᵣ—R₁₇), —(CH₂)ᵣNH—SO₂—(CH₂)ᵣ—R₁₅, —(CH₂)ᵣNH—CO—N(R₁₆)((CH₂)ᵣR₁₇), —(CH₂)ᵣNH—CS—N(R₁₆)((CH₂)ᵣ—R₁₇), —CO—NH—(CH₂)ᵣ—C₃-C₆ cycloalkyl and —CO—NH—(CH₂)ᵣ-aryl; and
R₁₄ is selected from the group consisting of optionally substituted straight or branched C₁-C₃ alkyl, optionally substituted aryl, optionally substituted heteroaryl, and optionally substituted heterocyclyl.

20. A compound according to claim 18, wherein m, p, q and t are each 0.

21. The compound according to claim 20, wherein n is 1 or 2.

22. A compound according to claim 20, wherein n is 0.

23. A compound according to claim 20, wherein R₁₄ is optionally substituted aryl.

24. A compound according to claim 20, wherein R₁₄ is optionally substituted 5 or 6-membered heteroaryl.

25. A compound according to claim 18, wherein each r is zero.

26. A compound according to claim 18, wherein R₁₀ is selected from the group consisting of —(CH₂)ᵣSO₂—N(R₁₆)((CH₂)ᵣ—R₁₇) and —(CH₂)ᵣNH—CO—(CH₂)ᵣ—R₁₅.

27. A compound according to claim 18, wherein R₁₀ is selected from the group consisting of —(CH₂)ᵣ—COO—(CH₂)ᵣ—R₁₅, —(CH₂)ᵣN(R₁₆)((CH₂)ᵣ—R₁₇), —(CH₂)ᵣR₁₆—(CH₂)ᵣR₁₇, —(CH₂)ᵣ—CO—N(R₁₆)((CH₂)ᵣ—R₁₇), —(CH₂)ᵣSO₂—(CH₂)ᵣ—R₁₅, and —(CH₂)ᵣSO₂—N(R₁₆)—CO—((CH₂)ᵣ—R₁₇).

28. A compound according to claim 19, wherein R₁₀ is —(CH₂)ᵣNH—SO₂—(CH₂)ᵣ—R₁₅, —(CH₂)ᵣNH—CO—N(R₁₆)((CH₂)ᵣ—R₁₇), —(CH₂)ᵣNH—CS—N(R₁₆)((CH₂)ᵣ—R₁₇), (CH₂)ᵣN—(SO₂R₁₆)(SO₂R₁₇), —CO—NH—(CH₂)ᵣ—(C₃-C₆ cycloalkyl) and —CO—NH—(CH₂)ᵣ-(aryl).

29. A compound according to claim 19, having a structure selected from the group consisting of:

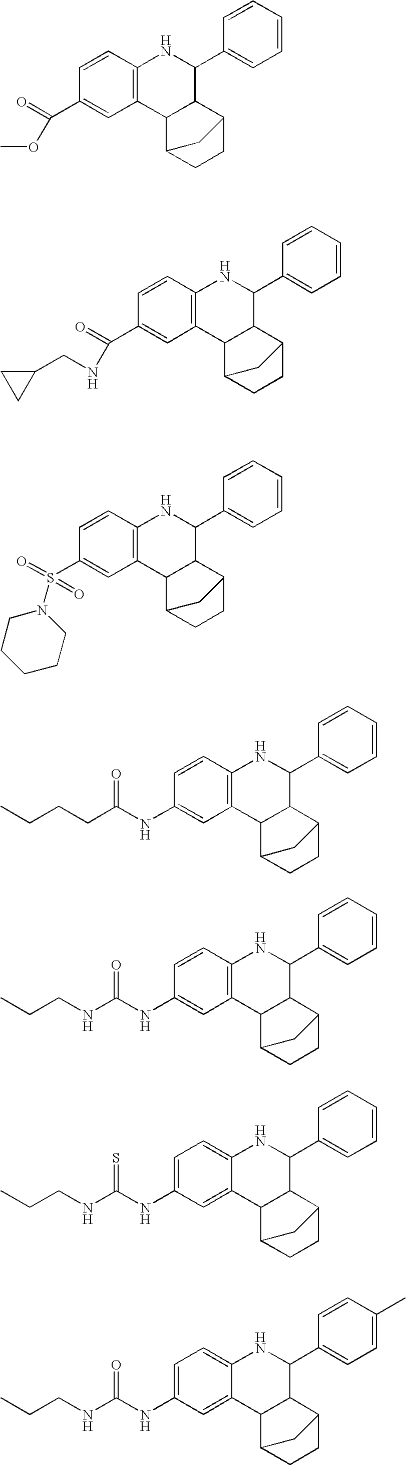

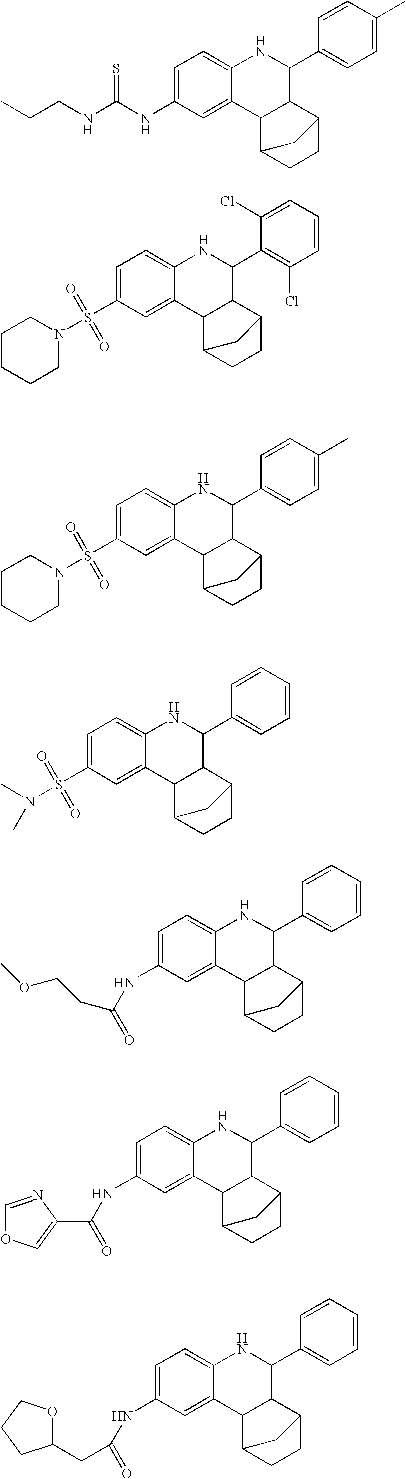

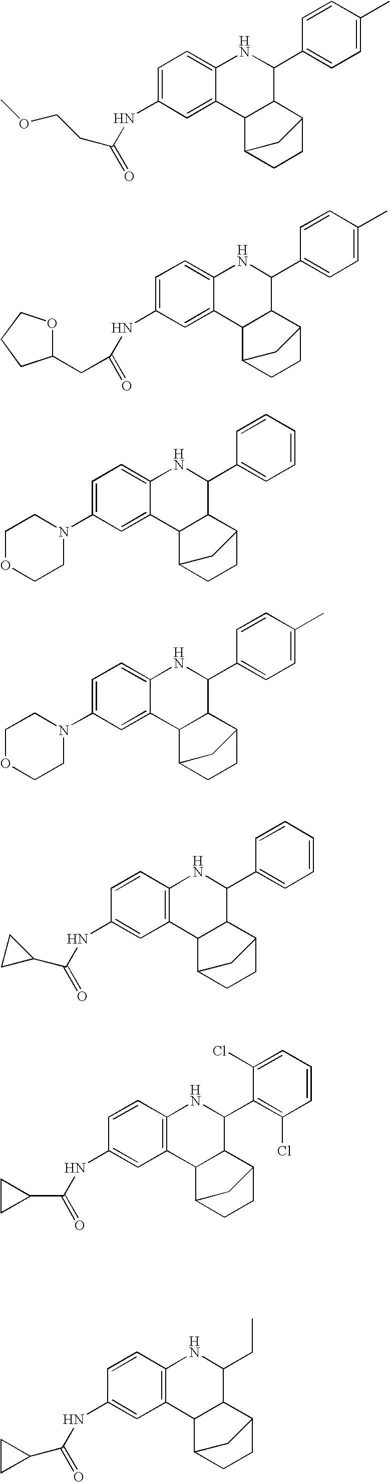

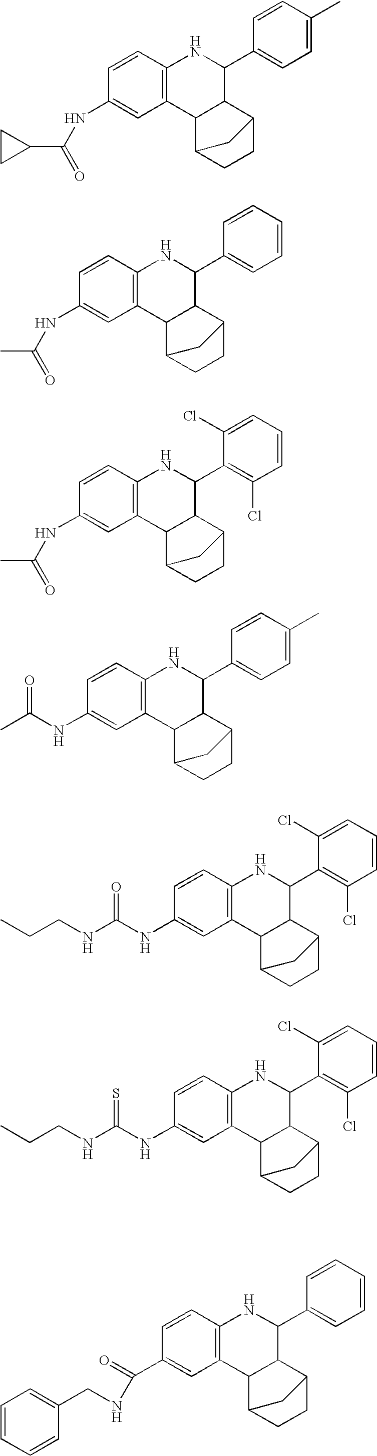

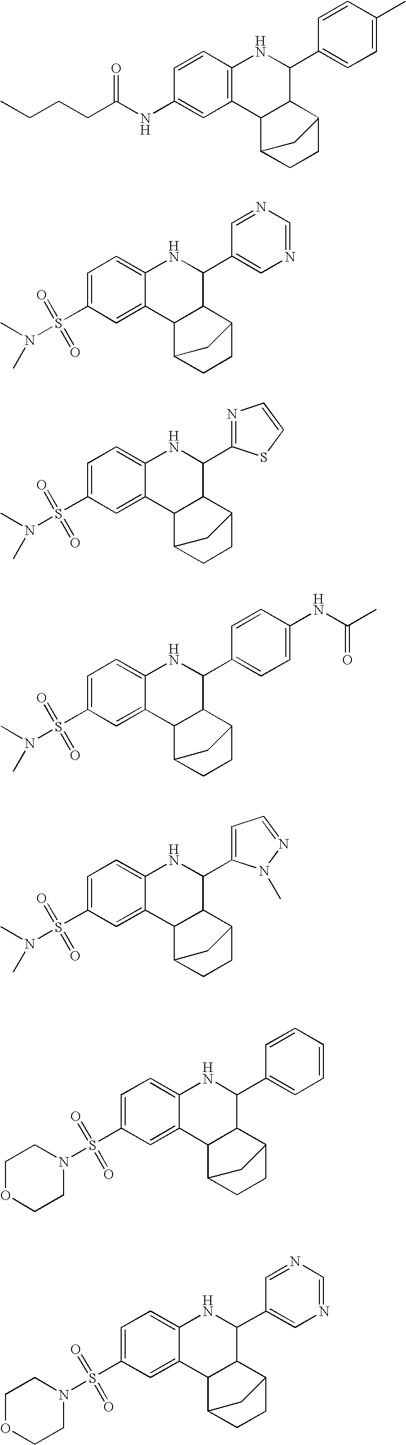

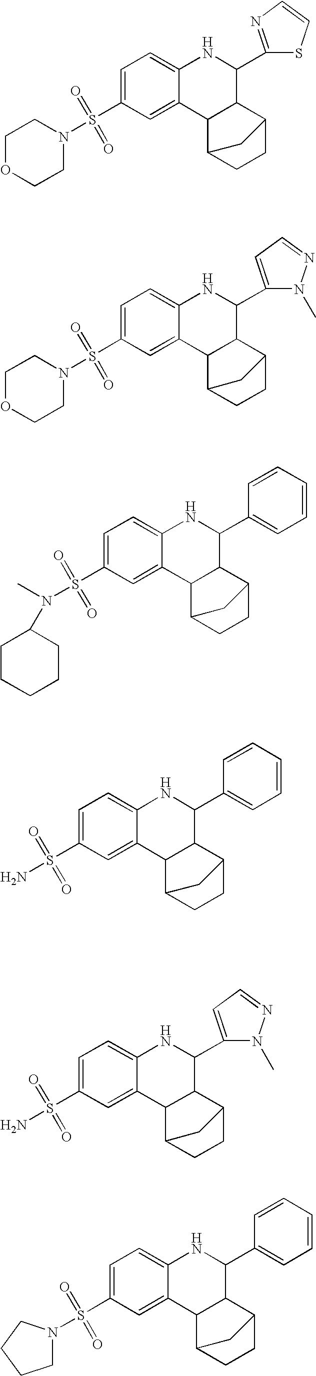

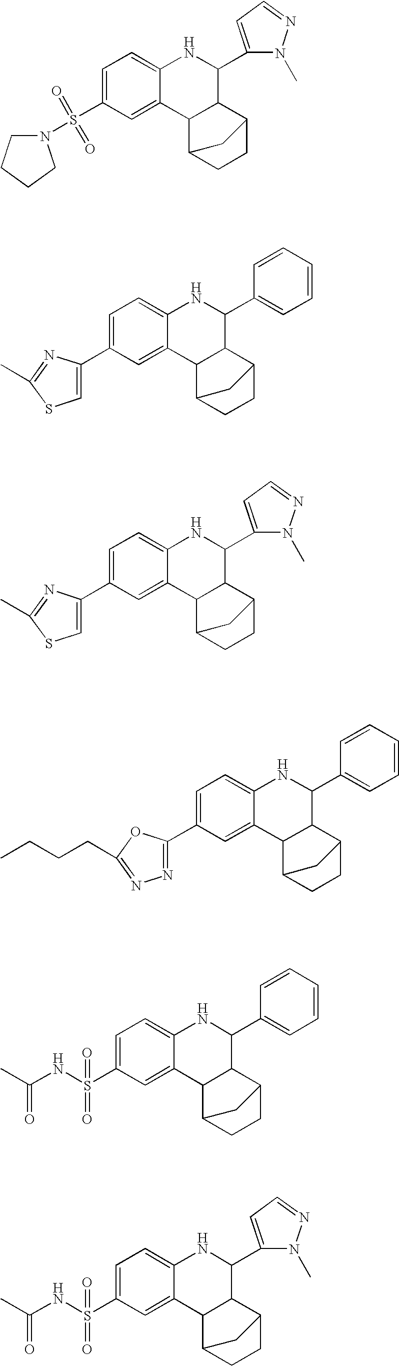

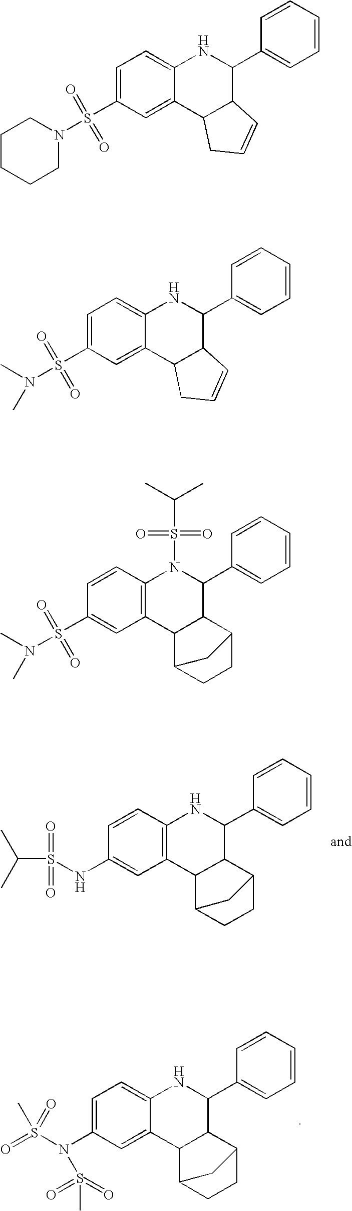

30. A compound according to claim 29, having the structure

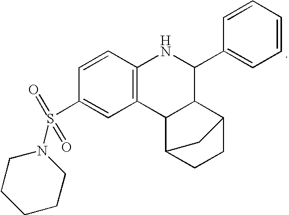

31. A compound according to claim 29, having the structure

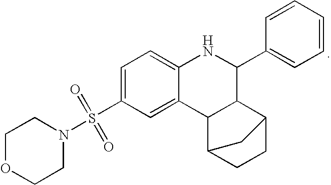

32. A compound according to claim 29, having the structure

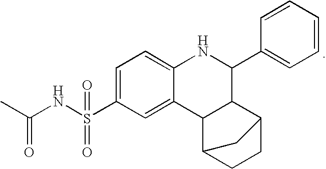

33. A compound according to claim 18, wherein the compound has an EC₅₀ of less than about 1 μM for the human cannabinoid-2 receptor.

34. A compound according to claim 33, wherein the compound has an EC₅₀ of greater than about 10 mM for the human cannabinoid-1 receptor.

35. A pharmaceutical composition comprising a compound of claim 18 and a pharmaceutically acceptable carrier, diluent or excipient.

36. A method of preventing or treating a CB2-associated disease or condition, the method comprising administering a pharmaceutical composition according to claim 35 to a patient in need thereof.
